(19) United States
(12) Patent Application Publication
(10) Pub. No.: US 2024/0203440 A1
(43) Pub. Date: Jun. 20, 2024

(54) Systems and Methods for Brain-Informed Speech Separation

(71) Applicant: The Trustees of Columbia University in the City of New York, New York, NY (US)

(72) Inventors: Nima MESGARANI, New York, NY (US); Enea CEOLINI, New York, NY (US); Cong HAN, New York, NY (US)

(73) Assignee: The Trustees of Columbia University in the City of New York, New York, NY (US)

(21) Appl. No.: 18/530,770

(22) Filed: Dec. 6, 2023

Related U.S. Application Data
(62) Division of application No. 18/129,469, filed on Mar. 31, 2023, now Pat. No. 11,875,813.
Application No. 18/129,469 is a continuation of application No. PCT/US2021/053560, filed on Oct. 5, 2021.
(60) Provisional application No. 63/087,636, filed on Oct. 5, 2020.

(51) Int. Cl. G10L 21/028; G10L 21/0208; G10L 21/0232

(52) U.S. Cl. CPC G10L 21/028; G10L 21/0232; G10L 2021/02087

(57) ABSTRACT

Disclosed are methods, systems, device, and other implementations, including a method (performed by, for example, a hearing aid device) that includes obtaining a combined sound signal for signals combined from multiple sound sources in an area in which a person is located, and obtaining neural signals for the person, with the neural signals being indicative of one or more target sound sources, from the multiple sound sources, the person is attentive to. The method further includes determining a separation filter based, at least in part, on the neural signals obtained for the person, and applying the separation filter to a representation of the combined sound signal to derive a resultant separated signal representation associated with sound from the one or more target sound sources the person is attentive to.

### CROSS-REFERENCE TO RELATED APPLICATIONS

This application is a divisional of U.S. application Ser. No. 18/129,469, entitled “SYSTEMS AND METHODS FOR BRAIN-INFORMED SPEECH SEPARATION,” and filed Mar. 31, 2023, which is a continuation application of, and claims priority to, International Application No. PCT/US2021/053560, entitled “SYSTEMS AND METHODS FOR BRAIN-INFORMED SPEECH SEPARATION,” and filed Oct. 5, 2021, which in turn claims priority to, and the benefit of, U.S. Provisional Application No. 63/087,636, entitled “BRAIN-INFORMED SPEECH SEPARATION (BISS) FOR ENHANCEMENT OF TARGET SPEAKER IN MULTI-TALKER SPEECH PERCEPTION” and filed Oct. 5, 2020, the content of which is incorporated herein by reference in its entirety.

### STATEMENT REGARDING FEDERALLY SPONSORED RESEARCH

This invention was made with government support under DC014279 awarded by the National Institute of Health. The government has certain rights in the invention.

### BACKGROUND

Hearing-impaired people often struggle to follow the speech stream of an individual talker in noisy environments. Recent studies show that the brain tracks attended speech and that the attended talker can be decoded from neural data on a single-trial level. Current speech separation solutions implemented in hearing aid devices include solutions based on array signal processing and beamforming. However, because the microphones are typically placed on the hearing aid itself, the efficacy of the beamforming solutions is limited by the small number of microphones and insufficient distance between them which is restricted by the size of the subject's head.

### SUMMARY

The present disclosure proposed a novel approach for speech enhancement and speaker separation (e.g., to mitigate the cocktail party problem) through a brain-informed speech separation (BISS) technique that combines speaker separation and speaker selection steps of auditory attention decoding (or AAD, which is a framework that uses neural signals to decode and enhance a target speaker in multi-talker speech perception). That is, information about the attended speech, as decoded from the subject's brain, is directly used to perform speech separation in the front-end. The approaches described herein use a deep learning model that uses neural data to extract the clean audio signal that a listener is attending to from a multi-talker speech mixture. This proposed framework can be applied successfully to the decoded output from either invasive intracranial electroencephalography (iEEG) or non-invasive electroencephalography (EEG) recordings from hearing-impaired subjects. It also results in improved speech separation, even in scenes with background noise. By jointly performing speech extraction and neural decoding, the neural signal directly guides a robust single channel speech extraction process/algorithm which is implemented using a neural network model. This method alleviates the need for a prior assumption of the number of speakers in the mixed audio and reduces the source distortion and computational load by extracting the target speaker from the scene. For these reasons, BISS represents a superior candidate for the implementation of a closed-loop, real-time, neuro-steered hearing aid (HA) which naturally adapts to different auditory scenes and number of competing sources.

Accordingly, in some variations, a speech separation method is provided that includes obtaining, by a device, a combined sound signal for signals combined from multiple sound sources in an area in which a person is located, obtaining, by the device, neural signals for the person, with the neural signals being indicative of one or more target sound sources, from the multiple sound sources, the person is attentive to, determining a separation filter based, at least in part, on the neural signals obtained for the person, and applying, by the device, the separation filter to a representation of the combined sound signal to derive a resultant separated signal representation associated with sound from the one or more target sound sources the person is attentive to.

Embodiments of the method may include at least some of the features described in the present disclosure, including one or more of the following features.

Determining the separation filter may include determining based on the neural signals an estimate of an attended sound signal corresponding to the one or more target sound sources the person is attentive to, and generating the separation filter based, at least in part, on the determined estimate of the attended sound signal.

Determining the estimate of the attended sound signal may include determining, using a learning process, an estimate sound envelope for the one or more target sound sources the person is attentive to.

Determining the separation filter may include deriving, using a trained learning model, a time-frequency mask that is applied to a time-frequency representation of the combined sound signal.

Deriving the time-frequency mask may include deriving the time-frequency mask based on a representation of an estimated target envelope for the one or more target sound sources the person is attentive to, determined based on the neural signals obtained for the person, and based on a representation for the combined sound signal.

The method may further include determining the estimated target envelope for the one or more target sound sources based on a machine-learned mapping process, implemented using regularized linear regression, applied to the obtained neural signals to produce the estimated target envelope.

Deriving the time-frequency mask may include combining the representation of the estimated target envelope with the representation for the combined sound signal to produce a fused signal.

Combining the representation of the estimated target envelope with the representation of the combined sound signal may include transforming the representation of the estimated target envelope into a 3D tensor estimated target envelope representation, transforming the representation of combined signal into a 3D tensor combined signal representation, and concatenating the 3D tensor estimated target envelope representation to the 3D tensor combined signal representation to generate a 3D tensor fused signal representation.

The method may further include processing the fused signal with a network of convolutional blocks arranged in a stack, wherein each of the convolutional blocks is configured to apply a convolutional process to input received from a respective preceding block, and to generate output comprising a sum of the input from the respective preceding block and output of the respective convolutional process applied to the input received from the preceding block.

The each of the convolutional blocks may include one or more convolution operators, at least one of the one or more convolution operators processing input data according to a dilation factor that is based on position of the respective convolutional block within the stack comprising the respective convolutional block.

The each of the convolutional blocks may further include one or more ReLU non-linearity elements.

The method may further include determining a time-frequency representation for the combined sound signal, including applying a short-time Fourier transform to the combined sound signal to generate a transformed combined sound signal, and compressing the transformed combined sound signal to generate a compressed spectrogram representation of the combined sound signal.

Applying the separation filter to the representation of the combined sound signal may include applying the time-frequency mask to the compressed spectrogram representation of the combined sound signal to generate an output spectrogram, and inverting the output spectrogram into a time-domain audio output signal.

The combined sound signal may include sound components corresponding to multiple receiving channels, and determining the separation filter may include applying multiple encoders to the sound components corresponding to the multiple receiving channels, with each of the encoders applied to each of the sound components, combining, for each of the multiple receiving channels, output components of the multiple encoders associated with respective ones of the multiple receiving channels, and deriving estimated separation functions based on the combined output components for each of the multiple receiving channels, each of the derived estimated separation functions configured to separate the combined output components for each of the multiple receiving channels into separated sound components associated with groups of the multiple sound sources.

The multiple receiving channels may include a first and second binaural receiving channels.

The combined sound signal may include representations of sound components corresponding to multiple receiving channels, and determining the separation filter may include applying multiple encoders to the representations of sound components corresponding to the multiple receiving channels, with each of the encoders applied to each of the sound components, determining spatial features based on the sounds components corresponding to the multiple receiving channels, combining the determined spatial features with output components of the multiple encoders associated with respective ones of the multiple receiving channels, to produce a combined encoded output, deriving, based on the combined encoded output, estimated separation functions, and separating, using the estimated separation functions, the combined encoded output into separated sound components associated with groups of the multiple sound sources.

Determining the spatial features may include determining one or more of, for example, interaural level difference (ILD) information, and/or interaural time difference (ITD) information.

The method may further include combining the separated sound components with the representations of the sound components to produce a combined enhanced signal representation, and deriving estimated separation functions based on the combined enhanced signal representation to separate the combined enhanced signal representation into separated enhanced sound components associated with the groups of the multiple sound sources.

The method may further include determining, based on the separated sound components, direction of arrival of the separated sound components.

Obtaining the neural signals for the person may include measuring the neural signals according to one or more of, for example, invasive intracranial electroencephalography (iEEG) recordings, non-invasive electroencephalography (EEG) recordings, functional near-infrared spectroscopy (fNIRS) recordings, and/or recordings captured with subdural or brain-implanted electrodes.

In some variations, a system is provided that includes at least one microphone to obtain a combined sound signal for signals combined from multiple sound sources in an area in which a person is located, one or more neural sensors to obtain neural signals for the person, with the neural signals being indicative of one or more target sound sources, from the multiple sound sources, the person is attentive to, and a controller in communication with the at least one microphone and the one or more neural sensors. The controller is configured to determine a separation filter based, at least in part, on the neural signals obtained for the person, and apply the separation filter to a representation of the combined sound signal to derive a resultant separated signal representation associated with sound from the one or more target sound sources the person is attentive to.

In some variations, non-transitory computer readable media is provided that includes computer instructions executable on a processor-based device to obtain a combined sound signal for signals combined from multiple sound sources in an area in which a person is located, obtain neural signals for the person, with the neural signals being indicative of one or more target sound sources, from the multiple sound sources, the person is attentive to, determine a separation filter based, at least in part, on the neural signals obtained for the person, and apply the separation filter to a representation of the combined sound signal to derive a resultant separated signal representation associated with sound from the one or more target sound sources the person is attentive to.

Embodiments of the system and the computer readable media may include at least some of the features described in the present disclosure, including at least some of the features described above in relation to the method.

Other features and advantages of the invention are apparent from the following description, and from the claims.

### BRIEF DESCRIPTION OF THE DRAWINGS

These and other aspects will now be described in detail with reference to the following drawings.

FIG. 1 is a schematic diagram of an example system implementing brain-informed speech separation.

FIG. 2A is a block diagram of a hint fusion module that may be included in a target extraction network shown in FIG. 1.

FIG. 2B is a schematic diagram of a partial arrangement of stacks which forms part of the example target extraction network shown in FIG. 1.

FIG. 2C is a diagram of an example block that may be used in any of the stacks shown in FIG. 2B.

FIG. 2D is a schematic diagram of an example configuration of the convolutional step/operator included is provided in FIG. 2C.

FIG. 2E is a schematic diagram of an example mask-generation module that is part of the example target extraction network shown in FIG. 1.

FIG. 3 includes a graph showing distribution of errors between the reconstructed attended envelope and the original attended envelopes for both EEG and iEEG.

FIG. 4 is a flowchart of an example sound separation procedure.

FIG. 5 includes violin plots of scale-invariant signal-to-distortion ratio (SI-SDR) illustrating SDR improvement from noisy speech mixture achieved from testing a brain-informed speech separation implementation.

FIG. 6 includes graphs showing envelope reconstruction results for iEEG recordings for an individual as a function of noise variance during curriculum training.

FIG. 7 includes two sets of graphs illustrating two examples of mask estimation test cases and results.

FIG. 8 includes a graph showing separation result performance of a brain-informed speech separation implementation for causal versus non-causal settings for the two subject groups.

FIG. 9 includes a graph showing the separation performance using envelopes reconstructed from EEG for each subject.

FIG. 10 includes a graph of distribution and the median SDR performance results for all individual subjects undergoing EEG tests.

FIG. 11 includes graphs with violin plots comparing performance of the BISS approach to a permutation invariant training (PIT) approach, for both causal and non-causal settings.

FIG. 12 includes graphs showing performance results for the implemented BISS framework when tested for a particular subject.

FIG. 13 includes a graph of distribution and median SDR performance results for all individual subjects of the EEG tests.

FIG. 14 is a schematic diagram of an example architecture for a multi-channel (e.g., binaural) speech separation network.

FIG. 15 is a schematic diagram of an example architecture for a binaural speech separation system for moving speakers.

Like reference symbols in the various drawings indicate like elements.

### DESCRIPTION

Disclosed are systems, methods, and other implementations (including hardware, software, and hybrid hardware/software implementations) directed to a framework called brain-informed speech separation (BISS) in which the information about the attended speech, as decoded from a subject's (listener's) brain, is directly used to perform speech separation in the front-end. Thus, in such embodiments, the neural signals are used in the filtering process applied to a received combined audio signal to obtain the audio signal of interest. Briefly, an AAD system (also referred to as a “brain decoder”) decodes an envelope (or some other representation) of the attended speech using brain signals (EEG or iEEG signals), and uses the decoded envelope (or “hint”) to incorporate that information into a deep-learning-based speech separation process/algorithm to provide information regarding which of the signals in the acoustic scene has to be extracted from a multi-talker speech mixture. The extracted (enhanced) speech of the desired speaker is then amplified and delivered to the user.

The framework described herein can be applied successfully to the decoded output from either invasive intracranial electroencephalography (iEEG) or non-invasive electroencephalography (EEG) recordings from hearing-impaired subjects. Other ways to measure neural signals may be used, including functional near-infrared spectroscopy (fNIRS) recordings, recordings through subdural electrodes, etc. The framework results in improved speech separation, even in scenes with background noise. The generalization capability of the system renders it a perfect candidate for neuro-steered hearing-assistive devices.

Accordingly, embodiments of the approaches described herein include a system that comprises at least one microphone to obtain a combined sound signal for signals combined from multiple sound sources in an area in which a person is located, one or more neural sensors to obtain neural signals for the person, with the neural signals being indicative of one or more target sound sources, from the multiple sound sources, the person is attentive to, and a controller in communication to the at least one microphone and the one or more neural sensors. The controller is configured to determine a separation filter based, at least in part, on the neural signals obtained for the person, and apply the separation filter to a representation of the combined sound signal to derive a resultant separated signal representation associated with sound from the one or more target sound sources the person is attentive to. In some examples, the controller configured to determine the separation filter is configured to derive, using a trained learning model implemented on the controller, a time-frequency mask that is applied to a time-frequency representation of the combined sound signal. In some embodiments, determining the separation filter may include determining based on the neural signals an estimate of an attended sound signal corresponding to the one or more target sound sources the person is attentive to, and generating the separation filter based, at least in part, on the determined estimate of the attended sound signal.

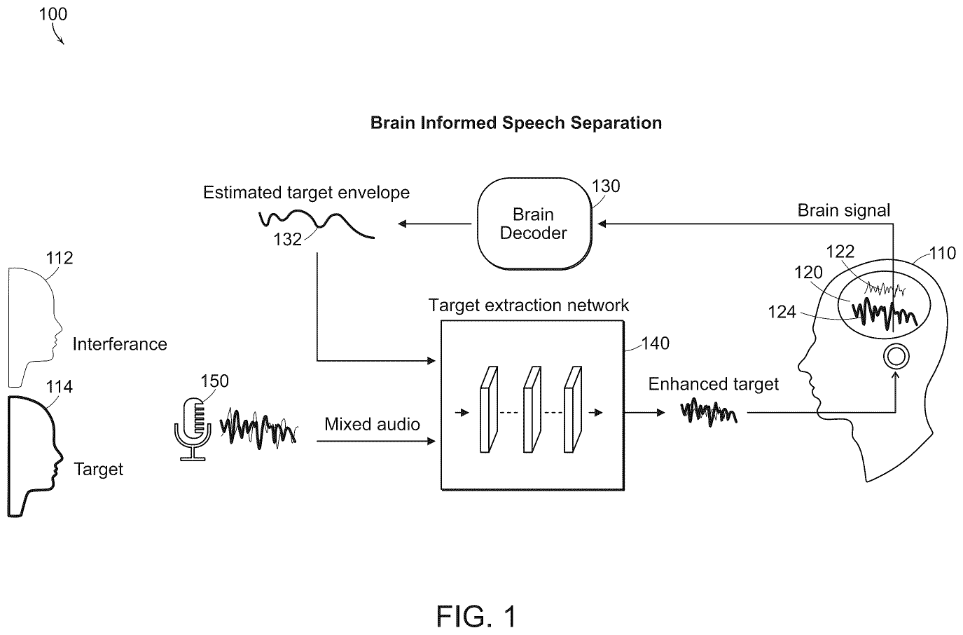

Thus, with reference to FIG. 1, a schematic diagram of an example system 100 implementing brain-informed speech separation is shown. As illustrated, a subject 110 attends to one (in this example, the lower target 114, in FIG. 1) out of two (or more) simultaneous talkers (the target 114 and a talker 112). The system includes one or more neural sensors (schematically represented as neural sensor 120) that are deployed on a surface of a hearing device secured to the head of the subject 110, or implemented as separate electrode in wired or wireless communication with the hearing device, to obtain neural signals 122 and 124 for the subject 110, based on which speech filtering (separation and/or other processing to extract the sound signal, from a mixed sound signal) is performed.

The measured neural signals 122 and 124 are delivered to a brain decoder 130 (which may be implemented using a processor-based device housed on the hearing device). In some examples, the decoding process is configured to estimate (e.g., via machine learning implementation) the envelope of the attended speech based on recorded brain signals. The recorded brains signal may include invasive intracranial electroencephalography (iEEG) recordings, non-invasive electroencephalography (EEG) recordings, functional near-infrared spectroscopy (fNIRS) recordings, recordings through brain-implanted and/or subdural electrodes, and/or other types of neural signal recordings acquired through appropriate sensors (e.g., electrodes secured externally to the subject, or implanted within the body of the subject, for example within the brain). The resultant output 132 of the brain decoder 130 (be it an estimated target envelope, or some other output signal representative of the sound signal that the subject is attending to or focusing on) is provided to a target extraction network 140, which may implement a speech separation neural network model, that receives, in addition to the decoded output signal 132, a speech mixture signal 152 generated by a microphone 150 (which may also be housed or deployed on the hearing device carried by, or secured to, the subject 110). The two inputs received, namely, the speech mixture, and the output signal 132 (a “hint” input, such as the decoded envelope) are used by the model implemented by the target extraction network 140 to separate and enhance the speech of the attended talker. The output of the model is the enhanced speech which is fed to the hearing aid device of the subject in this closed-loop setup. Thus, in the approaches of FIG. 1, the filtering processing performed by the target extraction network 140 is an adaptable process that adaptively configures the filtering (e.g., by generating a mask applied to a representation based on the combined audio signal) realized by the target extraction network 140 based on inputs that include the actual mixed signal that is to be separated, and the neural signals measured from the subject that provide information on who the subject 110 is attending to.

As noted, in some embodiments, the brain decoder 130 is configured to reconstruct the speech envelope of the attended speaker from the raw data collected by EEG or iEEG sensors. The decoder 130 may be implemented a spatio-temporal filter that maps the neural recordings (e.g., 122 and 124) to a speech envelope. The mapping may be based on a stimulus reconstruction method which may be learned, for example, using regularized linear regression or a deep neural network model. For both the EEG and iEEG data, a subject-specific linear decoder can be trained on S-T data and used to reconstruct speech envelopes on the M-T data. This approach avoids potential bias introduced by training and testing on the M-T data. For the iEEG data, only the outputs of a subset of electrodes may be used as input to the decoder. In such embodiments, electrode selection can be conducted via a statistical analysis to determine whether a specific electrode is significantly more responsive to speech compared to silence.

In some examples, a speaker-independent speech separation neural network model (such as that implemented by the network 140) is trained using the brain signals of the listener to guide the separation. As illustrated in FIG. 1, the two inputs to the speech separation neural network are the noisy audio mixture and the hint represented by the attended speech envelope decoded from the listener's neural signals. The audio mixture y(t) generally includes the sum of the attended speaker s_{d}(t) and all undesired sound sources sᵤ(t) (other speakers and noise) such that y(t)=S_{d}(t)+Sᵤ(t), where t represents the time index. The time-frequency representation of this mixture Y(l,f) can be obtained by taking the short-time Fourier transform (STFT) of y(t), specifically:

$$y ( l , f ) = S TFT ( y ( t ) ) = {S}_{d} ( l , f ) + {S}_{u} ( l , f ) ,$$

where l and f are time and frequency bin indices, respectively.

The complex mixture spectrogram Y∈ [symbol] ^{F×L} may be compressed, e.g., by a factor of 0.3, to reduce the dynamic range of the spectrogram; thus: Yc=(Y)^{0.3} where Y_{c}∈ [symbol] ^{F×L}.

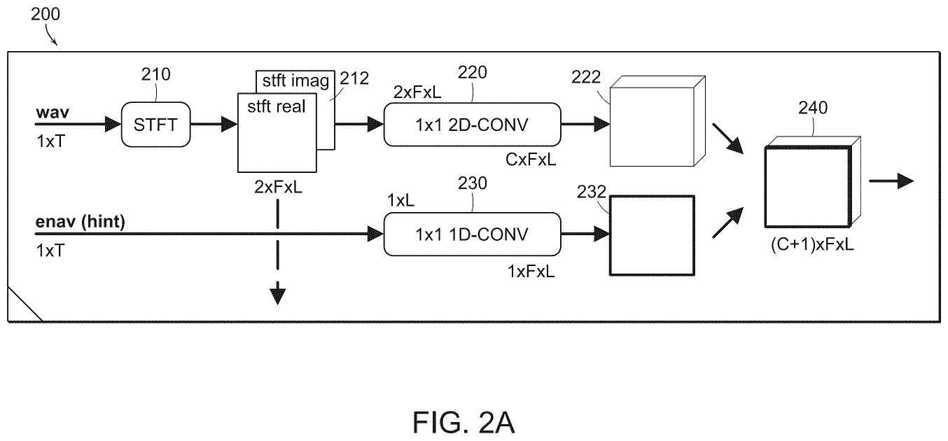

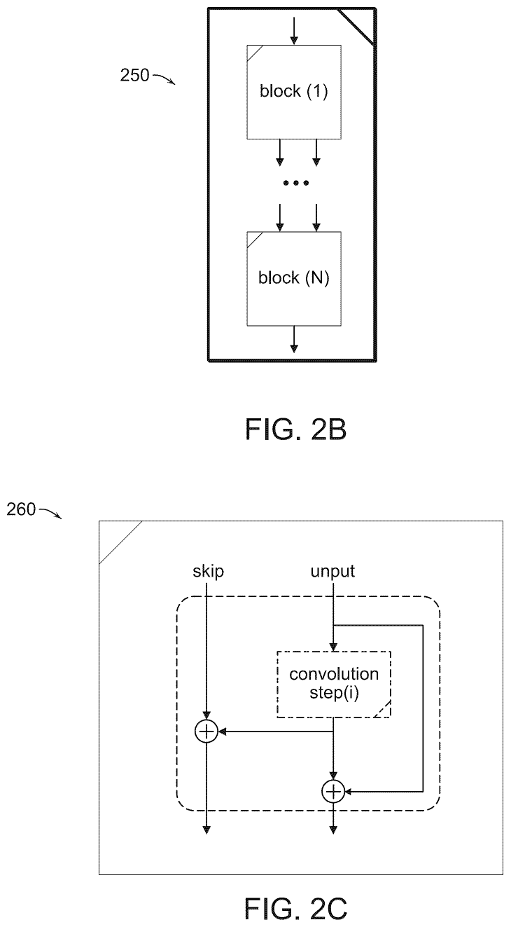

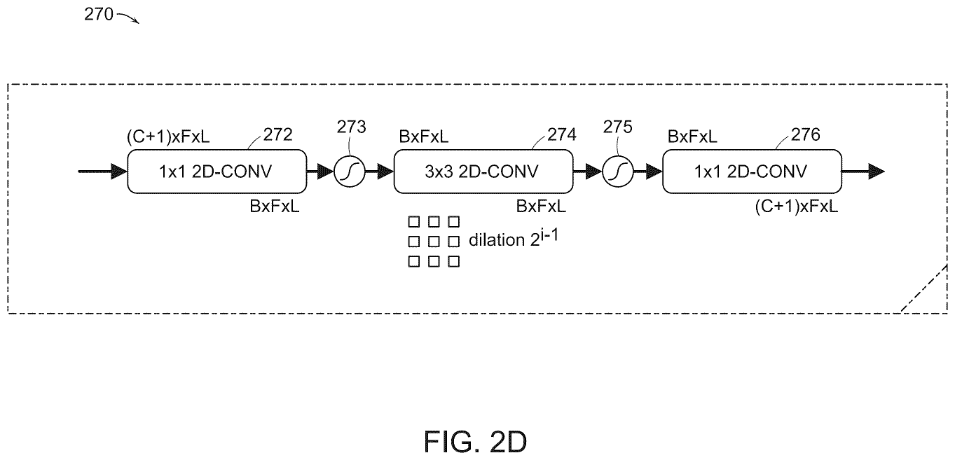

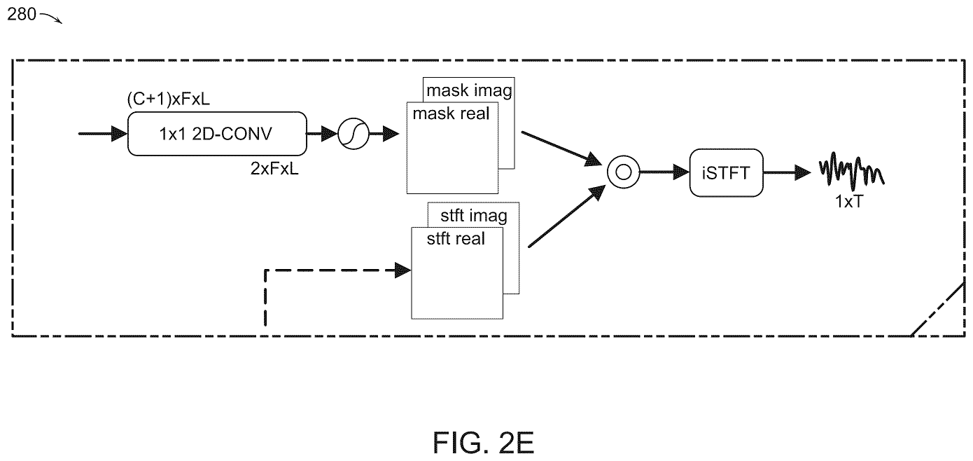

A Separation model implemented by the target extraction network 140 is realized, in some embodiments, based on an architecture that only uses 2D convolution structure (but possibly may use other configurations and structures, including a long-short term memory (LSTM) network). The use of a 2D convolution architecture is motivated because processing is performed in the time-frequency domain. The use of convolutional layers allows to decrease the number of parameters in the model and to control the temporal length of the receptive fields. The general architecture includes a computational block that fuses a hint signal (as will be described in greater detail below in relation to FIG. 2A) with the mixture audio, followed by a processing arrangement that includes stacks of convolutional layers (each of which may be identical in its architecture and number of parameters, thereby making the architecture modular). A final block applies the estimated complex mask M to the compressed input mixture spectrogram Y^{c} and inverts the estimated output spectrogram to the time domain.

Although the example embodiments presented herein uses a trainable 2D convolutional architecture to produce separations filter (e.g., masks) to extract an attendant speaker's speech, or to determine a decoded brain signal representative of a brain-informed signal to be combined with the mixed sound signal, other types/configurations of artificial neural networks may be used in place of the embodiment described herein. Other types of learning engines that may be used to generate separation filters or decoded brain signal representations include, for example, recurrent neural network (RNN)-based implementations, which may be based on an LSTM encoder-decoder architecture. Additional learning network configurations include other types of convolutional neural networks (CNN), and feed-forward neural networks. Feed-forward networks include one or more layers of nodes (“neurons” or “learning elements”) with connections to one or more portions of the input data. In a feedforward network, the connectivity of the inputs and layers of nodes is such that input data and intermediate data propagate in a forward direction towards the network's output. Unlike an RNN configuration, there are typically no feedback loops or cycles in the configuration/structure of the feed-forward network. Convolutional layers allow a network to efficiently learn features by applying the same learned transformation(s) to subsections of the data. Other examples of learning engine approaches/architectures that may be used include generating an auto-encoder and using a dense layer of the network to correlate with probability for a future event through a support vector machine, constructing a regression or classification neural network model that predicts a specific output from data (based on training reflective of correlation between similar records and the output that is to be predicted), etc.

Neural networks and/or other types of machine-learning implementations can be implemented on any computing platform, including computing platforms that include one or more microprocessors, microcontrollers, and/or digital signal processors that provide processing functionality, as well as other computation and control functionality. The computing platform can include one or more CPU's, one or more graphics processing units (GPU's, such as NVIDIA GPU's, which can be programmed according to, for example, a CUDA C platform), and may also include special purpose logic circuitry, e.g., an FPGA (field programmable gate array), an ASIC (application-specific integrated circuit), a DSP processor, an accelerated processing unit (APU), an application processor, customized dedicated circuitry, etc., to implement, at least in part, the processes and functionality for the neural networks, processes, and methods described herein. The computing platforms used to implement the neural networks typically also include memory for storing data and software instructions for executing programmed functionality within the device. Generally speaking, a computer accessible storage medium may include any non-transitory storage media accessible by a computer during use to provide instructions and/or data to the computer. For example, a computer accessible storage medium may include storage media such as magnetic or optical disks and semiconductor (solid-state) memories, DRAM, SRAM, etc.

The various learning processes implemented through use of the neural networks described herein may be configured or programmed using, for example, TensorFlow (an open-source software library used for machine learning applications such as neural networks). Other programming platforms that can be employed include keras (an open-source neural network library) building blocks, NumPy (an open-source programming library useful for realizing modules to process arrays) building blocks, etc.

As noted, the separation of the mixed/combined signal is based, in part, on use of a hint signal, generated from measured neural signals, to produce a signal that represents speech of the attended speaker. The hint input (e.g., the decoded envelope 132 representing what the subject is perceiving) may come from the temporal envelope of the clean speech of the attended speaker: h(t)=ls_{d}(t)|^{0.3}, where the absolute value of the waveform, s_{d}(t) is calculated and, in some embodiments, compressed by a factor of 0.3. During the training of the neural network model, the envelope is calculated from the clean audio signal.

In order to extract the speech of the desired speaker from the mixture, the speech separation neural network model is trained to estimate a complex valued mask M∈ [symbol] _{F×L}. The estimated mask M is applied pointwise to the input STFT Y_{c}, namely:

$${\hat{S}}_{d}^{c} = M ▯ {Y}^{c} .$$

The resulting estimated spectrogram is decompressed and inverted to the time domain to obtain an enhanced version of the desired speech ŝ_{d}. Specifically, the decompression operation produces Ŝ_{d}=(Ŝ_{d}^{c})³, and the inversion operation produces

In some example implementations, audio signals processing may include capturing (through a single microphone, or through multiple microphones) audio segments (e.g., 4 seconds segments) that are transformed to the frequency domain with a STFT using a window size of 512 and a step size of 125. The choice of the length in time (4 seconds) is arbitrary and different segment lengths may be used instead. The choice of 125 samples is appropriate for some applications because the audio sampling rate is 8 kHz and an output rate of 64 Hz, that matches the envelope sampling rate, may be desired. Because of the Hermitian property of the Fourier transform on real data, only the positive frequencies of the transformed signal can be kept, thus obtaining as input a 3D tensor of size 2×257×257. For the output mask, a complex-valued mask may be used instead of a real-valued magnitude mask. Using a real-valued magnitude mask forces the use of the noisy phase when inverting the estimated separated spectrogram to the time domain, and it has been shown that using the compressed complex mask gives better results. Because, in some embodiments, a complex STFT with overlapping windows is used, there exists an ideal complex mask that perfectly isolates the desired source from the mixture. Unfortunately, the mask values can be arbitrarily high and unbounded, and this poses a problem for the training process. For this reason, a hyperbolic tangent compression may be used that limits the output mask values to the range [−1, 1]. In such situations, only an approximation of the ideal mask can be computed.

As noted, to incorporate information about neural signals into the mask-generating process, a hint fusion procedure is implemented (and may be part of the target extraction network 140 of FIG. 1). With reference to FIG. 2A, a block diagram of a hint fusion module 200 is depicted. The hint fusion procedure includes two different processing steps that allow concatenating the audio waveform of the mixture Ye with the desired speech envelope H(l). First, the mixture waveform is transformed in the frequency domain by means of an STFT unit 210. The real and imaginary parts are then concatenated along a new axis effectively producing a 3D tensor 212 of size 2×F×L. A 1×1 2D convolution with C feature maps is then applied (at block 220) to obtain a 3D tensor 222 of shape C×F×L. Similarly, the desired (attended) speech envelope is processed with a 1×1 1D convolution unit 230 and expanded to become a 3D tensor 232 of shape 1×F×L. Finally, the two tensors are concatenated along the feature map axis to obtain a 3D tensor 240 of shape (C+1)×F×L.

The network realizing the hint fusion module 200 also includes an arrangement 250 of S stacks (illustrated in FIG. 2B), with each stack (individually indexed as stack s) being composed of multiple blocks. An example block 260 (each block is index block i) used in a stack is provided in FIG. 2C. The block 260 receives two inputs: the skip connection (r) from the input and the output (o) of a previous block. The skip connection is the sum of the input plus the output of each convolutional step, while the output of the block is the output of the convolution summed with the residual connection of the current input. This implementation can be expressed as:

$${p}_{i}^{s} = {c}_{i}^{s} + {s}_{i - 1}^{s} , and$$

$${o}_{i}^{s} = {o}_{i - 1}^{s} + {c}_{i}^{s}$$

Generally, the skip input to the first block in a stack is a matrix of zeros, while the output of the last block, and thus of the stack, is the skip path. Each block contains a convolutional step unit, such as the example convolutional step unit 270 depicted in FIG. 2D. In some embodiments, the convolutional step unit for all blocks (of all stacks) may have the same architecture, but may vary by having different dilation factors that are defined by the block index i. For example, the dilation factor for block i may be set to 2′. In some embodiments, the convolutional step has three parts: a) a 1×1 convolution operator 272 followed by a ReLU non-linearity element 273, b) a 3×3 convolution element 274 with a dilation factor i followed by a ReLU non-linearity 275, and c) another 1×1 convolution element 276. These parts can be represented as follows:

$${b}_{i , 1}^{s} = Re LU ( {conv}_{i , 1} ( {o}_{i - 1} ) ) ,$$

$${b}_{i , 2}^{s} = Re LU ( {conv}_{i , 2} ( {b}_{i , 1}^{s} ) ) , and$$

$${p}_{i , 3}^{s} = {c}_{i}^{s} = {conv}_{i , 3} ( {b}_{i , 2}^{s} ) ) .$$

The final convolutional step is utilized to get back the same input shape which allows the residual and skip connections to be added. This step increases the total number of parameters in the network without increasing the receptive field. Batch norm is applied at the end of the convolutional step. Overall, the receptive field (RF) in both frequency and time can be calculated as follows:

$$RF ( N , S , k ) = k + S {\sum }_{i = 0}^{N} ( k - 1 ) {2}^{i}$$

where k is the kernel size.

Square kernels are used so the receptive fields have the same dimension in both the frequency and time domain in terms of bins, but are different in terms of meaning and measure.

The last step of the extraction network implementation/process is the mask-generation module 280, schematically depicted in FIG. 2E. As shown, the output of the last stack, o_{N}^{s} is reshaped by a 1×1 convolution from a shape of (C+1)×F×L to a shape of 2×F×L, where the first dimension represents the concatenation of real and imaginary parts. In some embodiments, the mask, M, is obtained by first applying a hyperbolic tangent to the output of that convolution and then summing real and imaginary parts properly. Thus:

$$\tilde{M} = tanh ( conv ( {o}_{N}^{S} ) ) , and$$

$$M = \tilde{M} ( 0 , : , : ) + i \tilde{M} ( 1 , : , : )$$

where the operation (j,:,:) represents the tensor slicing that selects only the j^{th} element in the first tensor dimension, and i represents the imaginary unit. The generated mask M is then applied to the combined audio signal to separate the desired speech. The model is relatively simple and has very few parameters (e.g., around half a million for the implementations used to obtain the results discussed in greater detail below).

Thus, the BISS system illustrated in FIG. 1 uses a representation (e.g., a decoded speech envelope) of neural signals corresponding to speech information perceived by a subject as the informed input to the speech separation network. Ideally, a neural network could be trained with the brain-decoded envelopes. However, the EEG and iEEG data collected for attention decoding typically amounts to less than one hour of data for each subject. This amount of data is not enough to train an accurate speech separation model which has millions of parameters (such a model would require in the order of tens of hours of recorded speech). To address this problem, the training of the speech separation model is decoupled from the training of the brain decoder model. The separately trained models are then fused at test time. In order to do this, the speech separation model is trained with the ground truth speech envelope extracted from the audio using same envelope calculation as those used for attention decoding model. This guarantees that the attention decoding model will provide an envelope which is most correlated with the desired speech to extract. In the tested implementations discussed herein, most of the EEG data was collected in Denmark using Danish audiobooks, while the iEEG data was collected in New York using English audiobooks. Since a single model is being proposed to extract desired speech from either EEG or iEEG, the training dataset for the speech separation model includes a mixture of English and Danish utterances (i.e., the model is not language-specific). The English materials used for training included the Wall Street Journal (WSJ) utterances in the WSJ-mix2 dataset often used for source separation benchmarks. The Danish utterances were taken from Danish audiobooks used for the EEG study. It is to be noted that the training data is completely separated from the testing data, i.e., the audio tracks used in the attention decoding for both EEG and iEEG are not part of the training dataset. The overall training dataset used for the tested implementations comprised 22 hours of data. Mixed sentences were created on-the-fly at training time as a data augmentation method to effectively increase the amount of data used in training.

When estimating the frequency-domain masks for speech separation, the mean squared error (MSE) is generally used as the cost function. However, the estimated masks are usually smeared, limiting the separation quality. In the approaches described herein, a time-domain optimization method is proposed for use with a frequency domain solution by embedding both the STFT and iSTFT procedure into the training pipeline. Because these operations are differentiable, the normal backpropagation algorithm can be used to train the model. An example of a cost function used to optimize the model is SI-SDR. Optimizing the SI-SDR has shown very good results in time domain separation due to the fact that the model directly optimizes the measure which is used to evaluate its performance. The SI-SDR metric (SDR for simplicity) can be calculated directly from the time domain signals as follows:

$${s}_{target} = \frac{〈 {\hat{s}}_{d} , {s}_{d} 〉 {s}_{d}}{{‖ {s}_{d} ‖}^{2}} ,$$

$${e}_{noise} = {{s}^{⌢}}_{d} - {s}_{target} , and$$

$$SI - SDR = 10 {log}_{10} \frac{{‖ {s}_{target} ‖}^{2}}{{‖ {e}_{noise} ‖}^{2}} .$$

In some embodiments, the neural network model can be trained, for example, using the Adam optimizer with default settings and early stopping as a regularizer.

In some implementations, the speech separation model may be trained using a clean speech envelope calculated directly from the audio ground truth. However, the envelope estimated from either EEG or iEEG is not a perfect reconstruction of the original envelope. Generally, decoded envelopes have a Pearson's correlation r of <0.3 for EEG data and about 0.6 for iEEG data. Because of this, it is important that the speech separation model is robust to a noisy hint envelope. The distribution of the noise in the decoding process is therefore estimated, and the variance of this noise is extracted for both EEG and iEEG data. The noise has a Gaussian distribution with μ=0 and σ_{iEEG}=0.2 for iEEG, and σ_{EEG}=0.3 for EEG signals. After training the speech separation model with clean speech envelopes, the training is continued using a curriculum training technique in which the amount of noise injected into the training data increases continuously for a number of epochs. This training schedule has been shown to be optimal for training a model that is robust to a large range of input signal-to-noise ratio (SNR)s. A schedule may be used in which the σ of the added noise increases in steps of 0.05 from [0.05, 0.6].

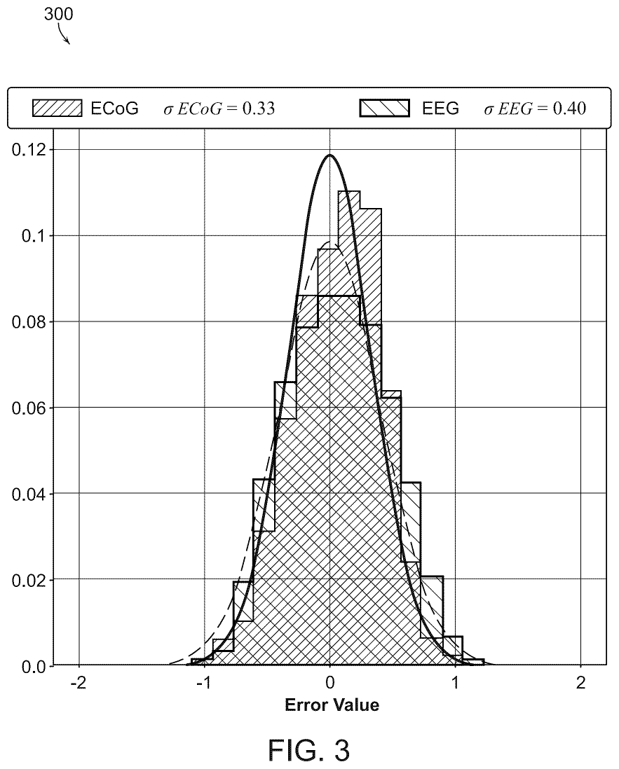

To make the speech separation model more robust to the degraded quality of the envelope reconstructed from the brain signals, a curriculum learning training scheme may be employed. This scheme includes increasing progressively, over training epochs, the difficulty of the task by introducing progressively more noise in the training. In order for this scheme to be effective, one needs to ensure that the noise injected during training is of the same distribution of the noise that will be present at test time. In some examples, an empirical distribution of the noise in the reconstructed envelope is used, which is represented by the error between the original envelope and the envelope reconstructed with AAD. This is exactly the noise that the network will be faced with when trained with the clean envelope and tested with the (noisy) reconstructed one. FIG. 3 includes a graph 300 showing the distribution of errors between the reconstructed attended envelope and the original attended envelope for both EEG and iEEG. As expected, the distribution of error for the EEG reconstruction has a bigger standard deviation with respect to the standard deviation of the iEEG reconstruction error.

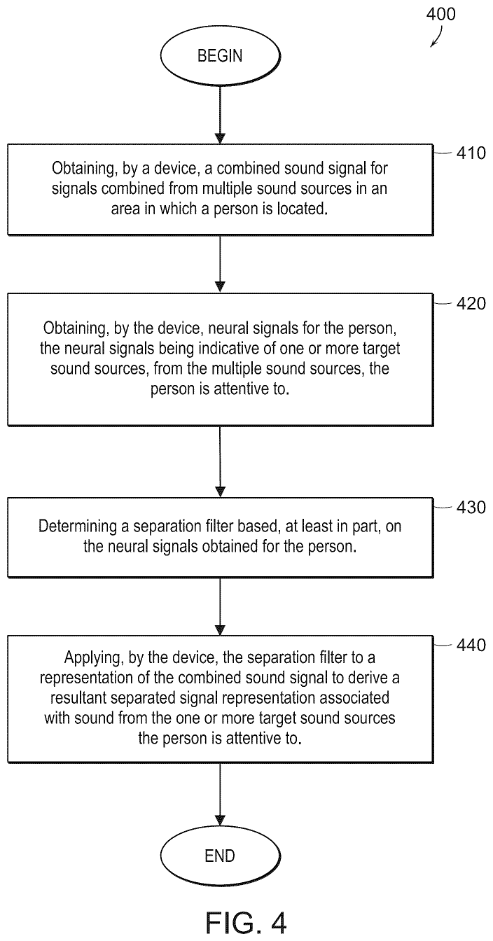

With reference next to FIG. 4, a flowchart of an example sound separation procedure 400 is shown. The procedure 400 includes obtaining 410, by a device (e.g., one or more microphones of a hearing device), a combined sound signal for signals combined from multiple sound sources in an area in which a person is located. The procedure 400 further includes obtaining 420, by the device, neural signals for the person, with the neural signals being indicative of one or more target sound sources, from the multiple sound sources, the person is attentive to. In some embodiments, obtaining the neural signals for the person may include measuring the neural signals according to one or more of, for example, invasive intracranial electroencephalography (iEEG) recordings, non-invasive electroencephalography (EEG) recordings, functional near-infrared spectroscopy (fNIRS) recordings, and/or recordings through subdural or brain-implanted electrodes.

With continued reference to FIG. 4, the procedure 400 further includes determining 430 a separation filter based, at least in part, on the neural signals obtained for the person, and applying 440, by the device, the separation filter to a representation of the combined sound signal to derive a resultant separated signal representation associated with sound from the one or more target sound sources the person is attentive to. Thus, in the approaches described herein, the neural signals are used not merely to indicate the target speaker the listener is focusing on, but to actually synthesize separation filters (e.g., in the form of a mask) that are applied to a combined signal (e.g., combining multiple signals captured, for example, by a single microphone or multiple microphones).

In some examples, determining the separation filter may include determining based on the neural signals an estimate of an attended sound signal corresponding to the one or more target sound sources the person is attentive to, and generating the separation filter based, at least in part, on the determined estimate of the attended sound signal. Determining the estimate of the attended sound signal may include determining, using a learning process, an estimate sound envelope for the one or more target sound sources the person is attentive to.

In some embodiments, determining the separation filter may include deriving, using a trained learning model (e.g., implemented on the target extraction network 140), a time-frequency mask (mask M discussed herein) that is applied to a time-frequency representation of the combined sound signal. As noted, the separation filter (in this example, a mask) may be based on the measured neural signals which indicate which speaker (or group of speakers) the subject is attentive to. An example of a derived representation of which speaker the listener is attentive to is to use a signal envelope, derived from neural signals (e.g., through a learning model) for the speech signal that the listener is focusing on. In such embodiments, deriving the time-frequency mask may include deriving the time-frequency mask based on a representation of an estimated target envelope for the one or more target sound sources the person is attentive to (with the estimated target envelope determined based on the neural signals obtained for the person), and further based on a representation for the combined sound signal. It is to be noted that other representations associated with the target speaker may be used. In examples in which the separation mask is derived based on an estimated target envelope, the procedure 400 may further include determining the estimated target envelope for the one or more target sound sources based on a machine-learned mapping process, implemented using regularized linear regression, applied to the obtained neural signals to produce the estimated target envelope.

In some examples, deriving the time-frequency mask may include combining the representation of the estimated target envelope with the representation for the combined sound signal to produce a fused signal. For example, combining the representation of the estimated target envelope with the representation of the combined sound signal may include (as also depicted in FIG. 2A) transforming the representation of the estimated target envelope into a 3D tensor estimated target envelope representation transforming the representation of combined signal into a 3D tensor combined signal representation, and concatenating the 3D tensor estimated target envelope representation to the 3D tensor combined signal representation to generate a 3D tensor fused signal representation.

In some embodiments, the procedure 400 may further include processing the fused signal with a network of convolutional blocks arranged in one or more stacks, with each of the convolutional blocks being configured to apply a convolutional process to input received from a respective preceding block, and to generate output comprising a sum of the input from the respective preceding block and output of the respective convolutional process applied to the input received from the preceding block. The each of the convolutional blocks may include, in such embodiments, one or more convolution operators, with at least one of the one or more convolution operators processing input data according to a dilation factor that is based on position of the respective convolutional block within the stack comprising the respective convolutional block. Each such convolutional block may further include one or more rectified linear activation function (ReLU) non-linearity elements. An example of a configuration of a convolutional block is provided in FIG. 2D. Alternative ways to combine (integrate or fuse) a signal representation of the sound signal (e.g., speech signal) attended to by the listener and the combined sound signals from multiple sources, in order to produce a composite signal combining sound information and attended speaker information, may be implemented (including by interlacing samples of the fused signals, performing a filtering operation to produce a composite signal, etc.)

In some embodiments, the procedure 400 may further include determining a time-frequency representation for the combined sound signal. This may include applying a short-time Fourier transform to the combined sound signal to generate a transformed combined sound signal, and compressing the transformed combined sound signal to generate a compressed spectrogram representation of the combined sound signal. In such embodiments, applying the separation filter to the representation of the combined sound signal may include applying the time-frequency mask to the compressed spectrogram representation of the combined sound signal to generate an output spectrogram, and inverting the output spectrogram into a time-domain audio output signal.

The brain-information speech separation approaches described herein were implemented and tested to obtain further details about the performance and features of the brain-information speech separation approaches. Brain recordings data used in the implementations described herein included EEG recordings from 22 normal hearing (NH) and 22 age-matched hearing-impaired (HI) subjects (NH: mean age 63.0±7.1; HI: mean age 66.4±7.0). HI listeners had a sloping high-frequency hearing-loss typical of presbycusis (age-related hearing loss). In 48 trials of ≈50 sec each, sub-jects listened to stories read by either a single talker (S-T) (16 trials), or multi talkers (M-T) (one male, one female, 32 trials). In the M-T trials, the two speech streams were presented at the same loudness level to allow unbiased attention decoding. The two competing speech streams were spatially separated at ±90° using non-individualized head-related transfer functions. On each trial, the subjects were cued to attend to either the male or female talker and the attended target was randomized across the experiment. After each trial, the subjects responded to 4 comprehension questions related to the content of the attended speech. Both NH and HI listeners had accurate speech comprehension for both the single-talker (NH: 93.3%, HI: 92.3% correct) and two-talker conditions (NH: 91.9%, HI: 89.8% correct). Despite high accuracy on speech comprehension questions, listening difficulty ratings revealed that the HI listeners rated the two-talker condition as being significantly more difficult than NH listeners did. The recordings data also included iEEG data collected from three subjects undergoing clinical treatment for epilepsy at the North Shore University Hospital, New York. These subjects were implanted with high-density subdural electrode arrays covering their language dominant (left) temporal lobe with coverage over the superior temporal gyrus (STG). Similar to the EEG experiments, the subjects participated in two experiments, a S-T experiment and a M-T experiment. In both experiments, the subjects listened to stories read by two speakers, one male speaker and one female speaker. In the S-T experiment, the subjects listened to each speaker separately, and in the M-T experiment the subjects listened to the two speakers talking concurrently with no spatial separation, i.e., the voices were rendered by a single loudspeaker placed in front of the subject. During the M-T experiment, each subject was presented with 11 minutes and 37 seconds of audio, making the S-T experiment twice as long. In the M-T experiment the audio was separated into 4 blocks (segments). In each block, the subject was asked to focus their attention on only one speaker. At the end of each block the subjects were asked to repeat the last sentence of the attended speaker to ensure that they were indeed paying attention to the correct speaker. All the subjects performed the task with high accuracy and were able to report the sentence with an average accuracy of 90.5% (S1, 94%; S2, 87%; and S3, 90%). The envelope of the high-gamma power was used at each site as a measure of neural activation.

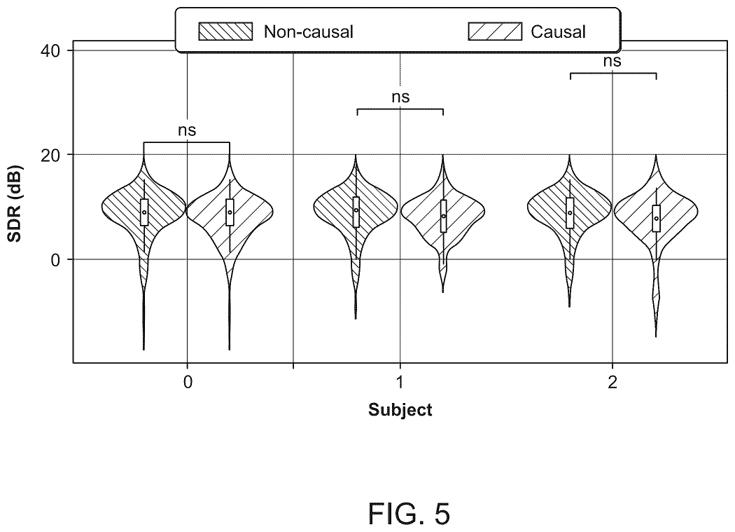

The BISS model described herein was tested on the iEEG recordings. FIG. 5 provides violin plots of scale-invariant signal-to-distortion ratio (SI-SDR) illustrating SDR improvement from the noisy speech mixture achieved from testing the BISS model with 4 s utterances. The results for the BISS framework were obtained for each subject separately, using envelopes decoded from the iEEG data, and for model settings of causal and non-causal (significance is indicated by ns if p>0.05 using Mann-Whitney U test). Each subject was tested on a set of 69 non-overlapping mixtures of two speakers for which SDR improvements using the clean reference signal were determined. The results presented in FIG. 5 show a comparable performance across all subjects. Subject 0 was the best with an SDR improvement of 9.5 dB; nevertheless, no significant difference between the scores of the three subjects was found. Additionally, the performance of causal and non-causal settings was similar for all subjects. One possible explanation for the similarity of performance across subjects is the noise training procedure in causal and non-causal settings. To test this hypothesis, the performances of the causal and non-causal models were tested using the noisy envelopes, like those used in training, rather than the neurally decoded envelopes provided as the hint (the brain information). The test showed a decrease in performances gap between the causal and non-causal settings from an initial 1 dB to 0.5 dB. This shows that while there might be a large difference in performance between causal and non-causal settings when using clean envelopes, this difference decreases when using noisy envelopes. This can explain the lack of significance between causal and non-causal settings in FIG. 5.

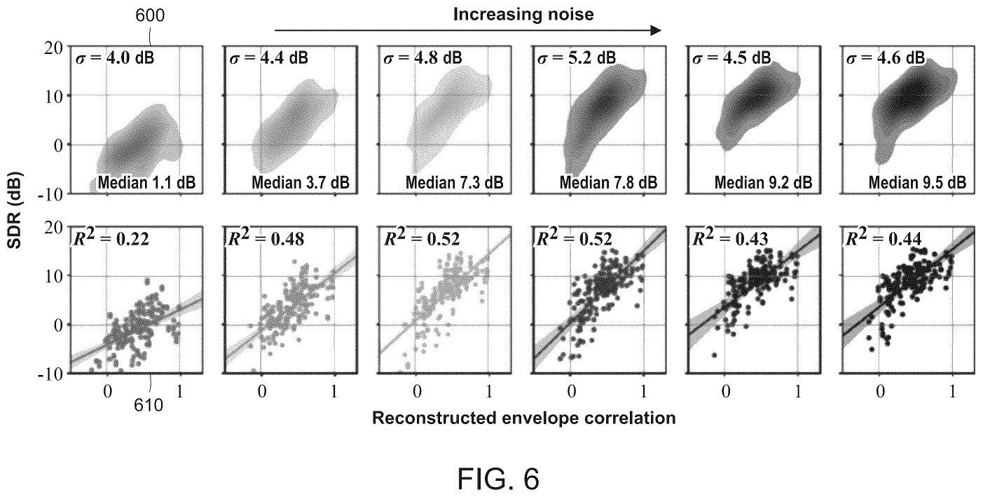

Next, the effects of the noise curriculum training on the model performance when utilizing neural data were investigated. FIG. 6 includes graphs showing envelope reconstruction results for iEEG recordings for an individual as a function of noise variance during curriculum training. The x-axis for the graphs indicates the r_{diff}=r_{attended}−r_{unattended} values, while the y-axis indicates SDR improvement in dB. The results shown in FIG. 6 were determined for 69 utterances when the individual (Subject 0) was attending to the male speaker in the mixture. The top panels of FIG. 6 show a density plot (using kernel density estimate with Gaussian kernels) of the utterances together with their median value, while the bottom panels show every single utterance plotted separately and a linear fit (using linear regression) of these points. The shaded areas in the plot represents the 95% confidence interval of the regression. Furthermore, the panels, going from left to right, show results from increasing the o of the noise during training (from σ=0.0 to σ=0.5 with steps of 0.1). The leftmost panels (e.g., graphs 600 and 610) show the results for the model without any noise training while the other panels show the effect of increasing the noise during training. The top panels additionally show that the median value shifts from below 0 dB, which indicates a failed separation, to above 9 dB, which indicates a very good separation. The bottom panels show that, independent of the noise level used in the training, there is a clear correlation between r_{diff} and the output SDR improvement. This indicates that the quality of the separation is linearly dependent on the quality of the envelope reconstruction in terms of Pearson's r value.

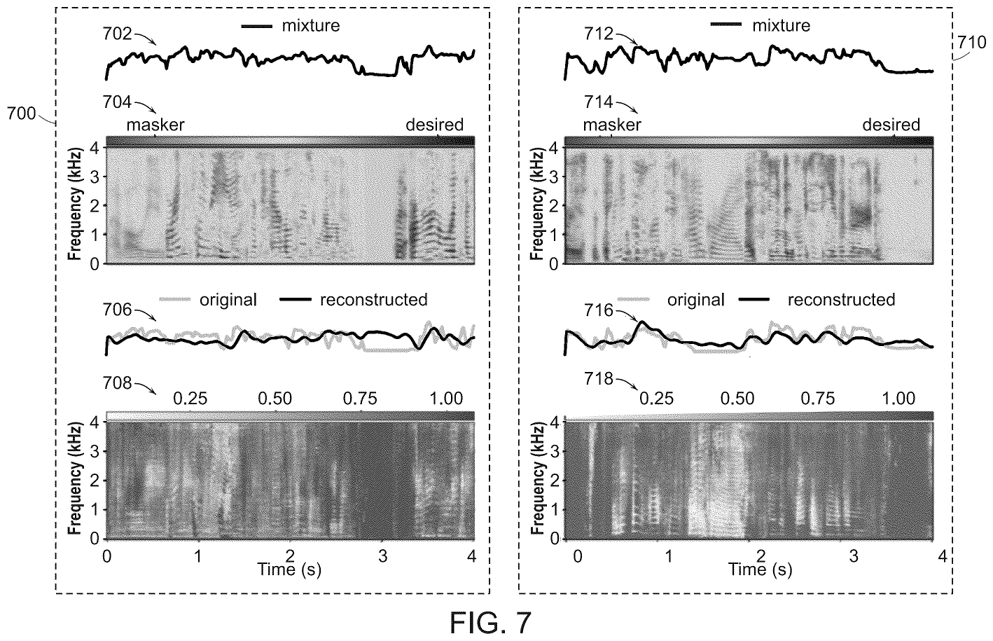

Next, the effect of using different Pearson's r values on the estimated mask M was explored. In particular, the investigation studied how the masks differ when an utterance with high correlation is compared to an utterance with low correlation. For example, FIG. 7 provides two sets of graphs, 700 and 710, illustrating two examples of mask estimation test cases and results. Each of the sets of graphs includes, from top to bottom, a mixture envelope (702 and 712), a mixture spectrogram (704 and 714) with desired speaker highlighted in darker shade, original and reconstructed desired speech envelopes (706 and 716), and mask estimated by the model based on the decoded envelope (708 and 718). The first example mask estimation test case (corresponding to the set 700) is of a failed mask with a correlation of −0.13 and an SDR improvement of −10.4 dB. The second example mask estimation test case (corresponding to the set 710) is of a successful mask with an r value of 0.69 and an SDR of 9.2 dB. The example mask estimation results of FIG. 7 shows that the mask for the failed utterance (corresponding to the set 700) has fewer sharp edges around the harmonics of the desired speech, while for the successful utterance (corresponding to the set 710) the mask is sharp around every part of the desired speech, and especially sharp around the harmonics. This is true even at smaller time scales where the sharpness of the mask tightly follows the correlation of the reconstructed envelope.

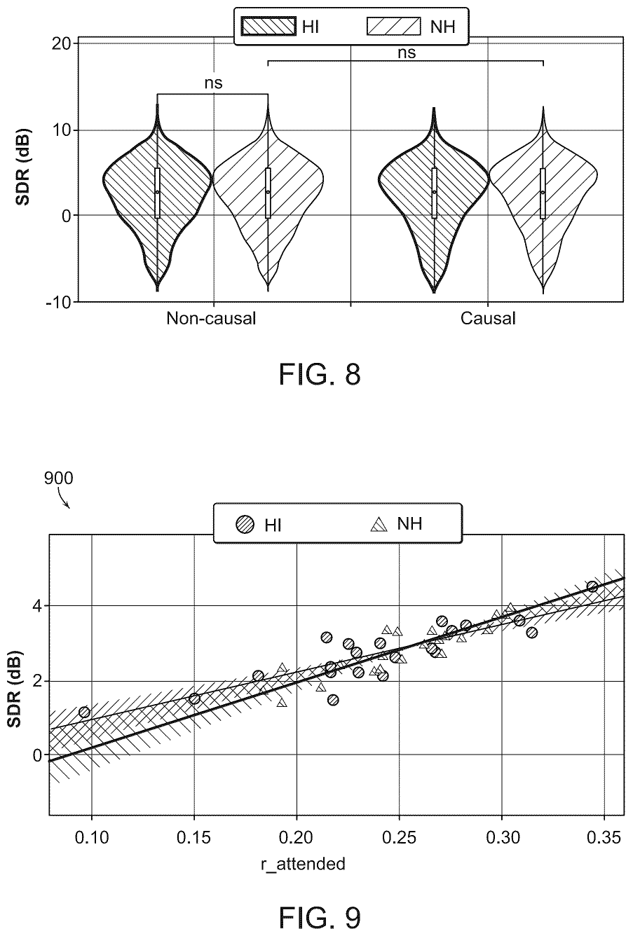

Turning next to the testing performed on the EEG dataset, the investigation focused mainly on the differences between NH (21 subjects) and HI (20 subjects) groups. For each subject, the performance was tested on 128 non-overlapping segments of 4 seconds. As in the iEEG case, the investigation looked at the differences in performance for the model under causal and non-causal settings. FIG. 8 shows the separation result performance of a brain-informed speech separation implementation for causal versus non-causal settings for the two subject groups. In the figure, the y-axis shows the separation quality in terms of SDR improvement in dB. Significance is indicated by ns if p>0.05 using Mann-Whitney U test.

As expected, the overall performance is lower for EEG than with iEEG. As with iEEG, no significant difference was found between the causal and non-causal settings (p=9.3e-01). Moreover, no statistical difference was found between NH and HI for the causal (p=4.508e-01) and non-causal settings (p=1.865e-01). The overall performance of each subject was also examined in terms of r_{diff} and SDR improvement. FIG. 9 includes a graph 900 showing the separation performance using envelopes reconstructed from EEG for each subject (the y-axis shows the separation quality in terms of SDR improvement in dB, and significance is indicated by ns if p>0.05 using Mann-Whitney U test). The graph 900 shows the median SDR versus the median r_{diff} for all EEG subjects. Similar to iEEG, both groups show a clear and similar correlation between the rain and SDR. Overall, the EEG results show a positive correlation with a slope of 14.2 which is very close to the overall positive correlation of iEEG data which is 14.7.

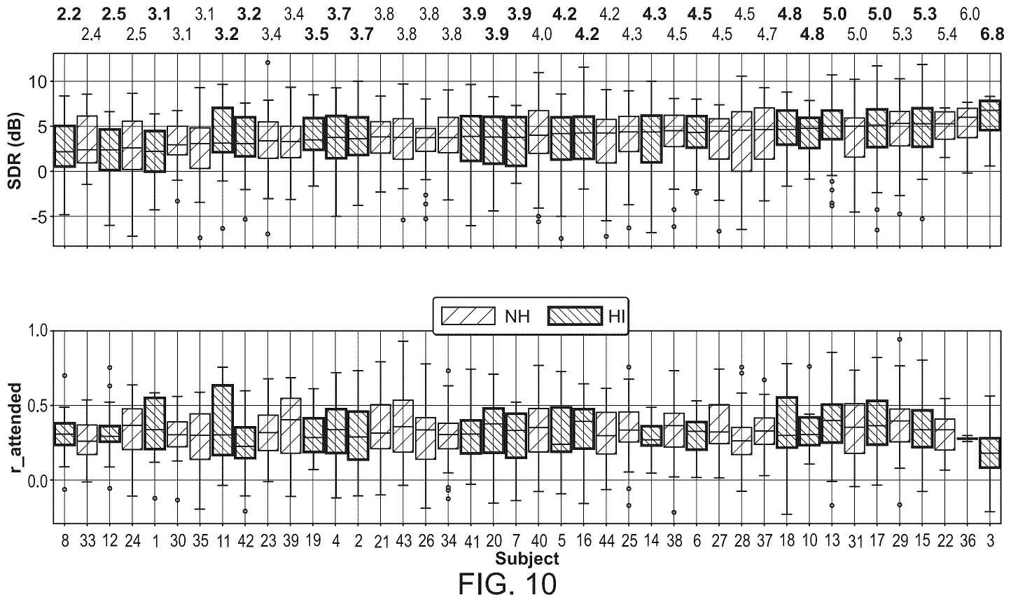

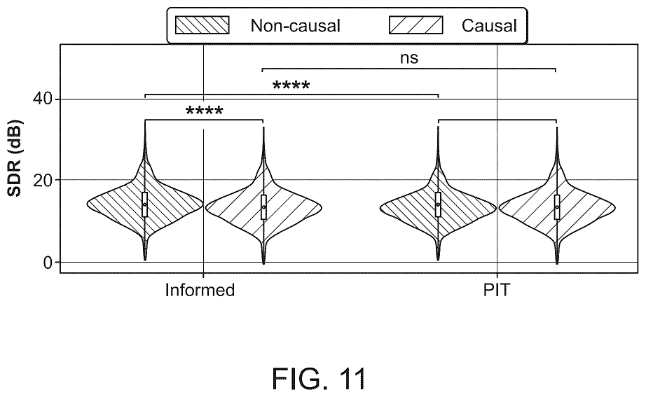

Additionally, the distribution of performance for each subject individually across the 128 utterances was examined. Only trials in which the decoding of utterances were successful were considered, i.e., with r_{diff}>0. FIG. 10 includes a graph 1000 of the distribution and the median SDR performance result for all individual subjects of the EEG tests, ordered by increasing SDR (median values for SDR are highlighted above the top panel). The difference in performance between the best and worst subjects is 4.6 dB, with the best and worst subjects having median SDRs of 6.8 dB and 2.2 dB, respectively.

The brain-controlled speech separation approach described herein is configured to use a single-trial neural responses of a listener attending to a speaker to extract and enhance that speaker from the mixed audio. By utilizing the information provided by the envelope reconstruction process/algorithm, this methodology can extract the attended speaker from a mixture of two (or more) speakers as well as from speech-shaped background noise in the auditory scene, making it a viable solution for neuro-steered hearing aids (HAs). Auditory attention decoding generally assumes that the clean speech of the speakers in a mixture is available to be compared to the neural signals to determine the target source. This access to clean sources is not realistic in real-world applications. The proposed novel framework combines the steps of speaker separation and speaker selection by turning speech separation into speech extraction. Not only does this framework readily generalize to competing speakers or background noise, but it also requires significantly less computation than other speech separation approaches because only the target speaker is extracted.

Specifically, as part of the testing and investigation of the performance of the BISS framework described herein, the proposed informed speech separation approach was compared with a permutation invariant training (PIT), which is a method for training deep-learning based speech separation models. In the testing conducted for the BISS approach, the hint input for the BISS model came from the envelope of the ground truth attended speech. FIG. 11 includes graphs with violin plots comparing performance of the BISS approach to the PIT approach, for both causal and non-causal settings. The results in FIG. 11 show that ISS gives significantly better results (p=7.8461e-09) than PIT for the causal setting. The non-causal setting results, on the other hand, show no significant difference (p=. 1101) between ISS and PIT. The ISS process, however, produces significantly better results under non-causal settings (p=5.0211e-09) over causal settings. The causal setting gives an absolute median difference of ˜ 0.9 dB, a value that still indicates good separation quality for practical applications. It is to be noted that the model trained with PIT has around 1 million parameters, while the model size scales almost linearly with the number of speakers in the mixture. On the other hand, the ISS model has only 0.5 million parameters and this number does not have to scale with the number of speakers in the mixture. Similarly, the number of operations to compute one spectrogram column mask is around 14 MOps for the PIT model and 7 MOps for the ISS model, which makes the ISS model more efficient and computationally cheaper so as to facilitate real-time applications. The number of parameters and number of operations are calculated based on the final settings of the model chosen for the best trade-off between size and performance. The final settings are shown in Table 1 and give rise to a receptive field with a span of 3.9 s in time and a span of 7900 Hz in frequency.

TABLE 1

| Symbol | Description | Value |
|---|---|---|
| F | Number of frequency bins | 257 |
| L | Number of STFT time | 257 |
| | windows | |
| T | Number of samples in the | 32000 |
| | waveform | |
| C | Channels in the stack | 32 |
| B | Channels in the convolutional | 64 |
| | step | |
| S | Number of stacks | 2 |
| N | Number of blocks | 6 |
| i | Index of each block (dilation | — |
| | factor) | |
| s | Index of each stack | — |
| k | kernel size | 3 |

Additionally, the speech separation quality (SDR) is highly correlated with stimulus reconstruction accuracy. This close correlation between these two quantities reveals two desired aspects of the proposed framework. First, it confirms the hypothesis that speech separation quality is higher in a model that takes additional information as input (see results of FIG. 11), in this case the target speaker envelope reconstructed from the neural responses of the listener. Moreover, it offers a more general solution with respect to speaker extraction since the information about the target speaker can be obtained directly from the subject's brain on a trial-to-trial basis and does not have to be known a priori. Second, the speech separation quality of the model in the proposed framework follows the attention level of the subject which directly affects the reconstruction accuracy (r_{diff}), and thus reflects the intent of the subject. In closed-loop applications of AAD, the separated target speech is typically added to the original mixed signal in order to both amplify the target speaker, but also to maintain the audibility of other sources to enable attention switching (usually 6-12 dB). Since the BISS framework creates an output SDR which is correlated with the attention of the subject (r), this alleviates the need to render the mixture speech with a particular SNR because the SNR will naturally reflect the attention of the subject. This attention driven target SNR could help with attention switching in closed-loop applications. The results obtained from applying AAD to EEG data are similar to the results obtained with iEEG but with smaller Pearson's r of the reconstructed envelope and lower SDR of separated speech. Even though these results are less accurate, they are in accordance with the predictions made using iEEG for AAD. In particular, the rain and the output SDR are highly correlated, confirming again that the model follows the subject's attention. Moreover, the AAD results using EEG show no significant difference in target speech enhancement (SDR) between HI and NH subjects. This shows that the proposed BISS approach can be used by HI subjects, which is a crucial aspect for the applicability of the framework to neuro-steered HAs.

It is also worth noting that the same speech separation model was used to produce the results presented from both iEEG and EEG. This shows the versatility of the proposed approach. Not only can the framework be applied successfully in the presence of different languages and noise, but it is also unaffected by different methods of reconstruction and different types of brain signals used. Particularly, to show that the BISS framework can successfully be applied across tasks of speaker separation and speech enhancement, the testing performed on the proposed framework also looked at the possibility of reducing noise in attended speech using EEG signals. This is an easier task to solve than speaker separation. This is mainly due to the fact that the noise and speech have different frequency distributions and are easier to separate than 2 overlapping speakers. In particular, speech enhancement models that use neural networks can easily be trained without the need to use PIT: if one assumes only one speaker, there is no mixed signal to resolve from which a desired signal is to be extracted. EEG recorded from a NH subject listening to speech in stationary speech-shaped background noise was used. The network tested is the same one used above, but it was trained with more added noise in the input, with respect to the model used for speaker separation. The hint to the network was still the envelope reconstructed from the EEG of the subject.

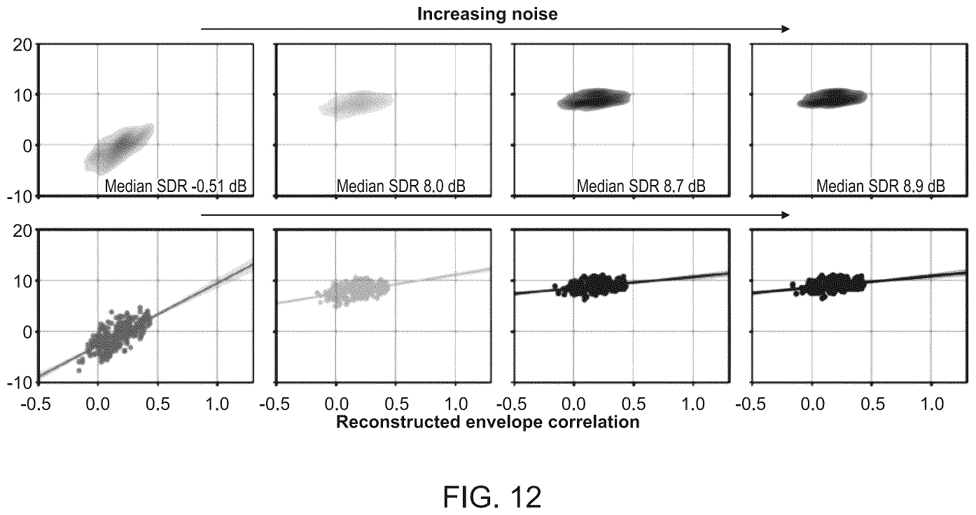

FIG. 12 includes graphs showing performance results for the implemented BISS framework when tested for a particular subject. In FIG. 12, the x-axis indicates r_{speech} value, and the y-axis indicates SDR in dB. The panels in the top row show the density distribution of the points using kernel density estimate with Gaussian kernels. The panels in the bottom row show each utterance separately and a linear fit obtained using linear regression. The shaded area represents the 95% confidence interval of the regression. The panels from left to right show results from increasing the o of the noise during training (from σ=0.0 to σ=0.6 with steps of 0.2).

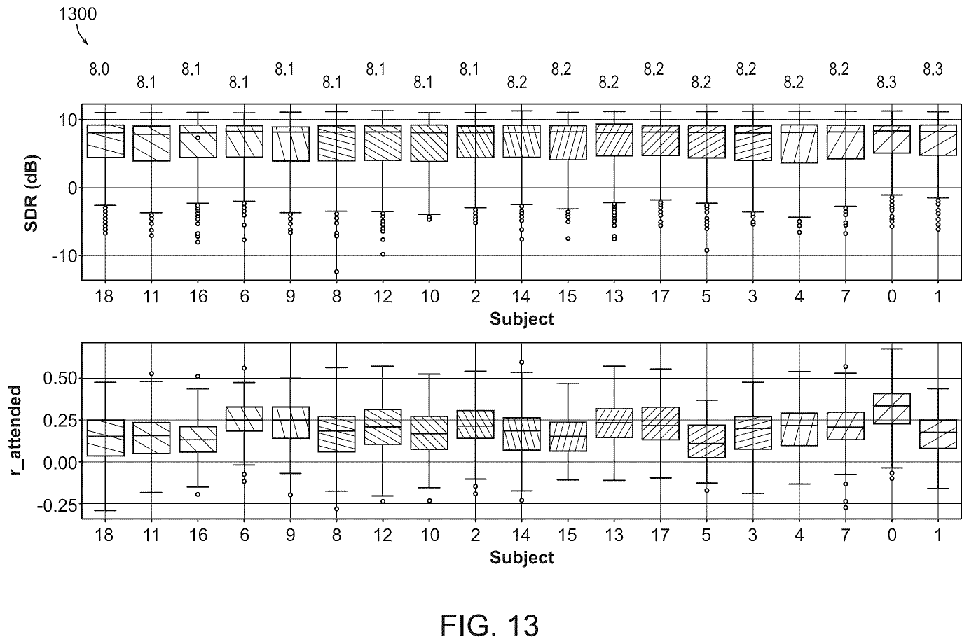

Results for the particular subject tested demonstrate that the training scheme is effective in increasing the robustness of the network to the non-perfect reconstructed envelope. As can be seen, compared to iEEG in speaker separation, even a low amount of noise helps the network in making use of the hint to separate the desired voice. Moreover, it can be seen from FIG. 13, which includes a graph 1300 of the distribution and the median SDR performance result for all individual subjects of the EEG tests, that the method can be successfully applied to all the subjects. Differently from the speaker separation task, it can be seen that for speech enhancement, the linear trend between Pearson's r value and output SDR is less evident than the one present for speaker separation. This is due to the fact that the task is much easier to solve and that even a reconstructed envelope with a low reconstruction quality is informative enough for the model to separate the desired speaker.

The above findings suggest that the BISS approach is a robust speech separation frontend. Moreover, the finding that BISS results in no significant difference between causal and non-causal speech separation models increases its usability in real-time systems which require causal, short-latency implementation (<20 ms).

Finally, BISS can decouple the optimization of front-end (speech separation) and back-end (AAD) systems even when a small amount of data is available. This joint optimization can also be done when large amounts of data are available. While the tested present approach used basic neural signal decoding (e.g., speech envelope reconstruction), there are many other ways to implement attention decoding, including, for example, by reconstructing the speech spectrograms. Moreover, the neural decoding can be done either with classification or state space models. These methods can be easily integrated into the BISS framework because it takes as the hint (speaker attending brain information) any signal that is correlated with the attended speech.

### ADDITIONAL EMBODIMENTS

The example separation technique (based on 2D convolutional operations) discussed in relation to FIGS. 1-13 is but one example of a separation technique in which brain-informed data can be leveraged to generate a separation filter(s) that is to be applied to a combined, multi-source sound signal. Other separations schemes may be used in place of, or in addition to, the sound/speech separation approach used in the implementations of FIGS. 1-13. Discussed below are additional examples of separation techniques in which the speaker attended information, determined from a listener's brain signals, can be used to determine and apply sound separation processing to extract the desired signal(s). These additional separation techniques can, for example, include a hint fusion module to create a composite signal from the captured multi-source signal and the speaker-attending information. Such a hint fusion module may be similar to, or different from, the hint fusion module 200 depicted in FIG. 2A.

A first additional example implementation of a separation technique that can be used in conjunction with, or as an alternative to, the separation systems described in relation to FIGS. 1-13 is one based on separating varying numbers of sources with auxiliary autoencoding loss. Iterative separation methods offer flexibility in that they can determine the number of outputs. However, such iterative techniques typically rely on long-term information to determine the stopping time for the iterations, which makes them hard to operate in a causal setting. Additionally, such techniques lack a “fault tolerance” mechanism when the estimated number of sources is different from the actual number. To mitigate these problems, a simple training method, the auxiliary autoencoding permutation invariant training (A2PIT) is proposed. A2PIT assumes a fixed number of outputs and uses auxiliary autoencoding loss to force the invalid outputs to be the copies of the input mixture. This methodology therefore detects invalid outputs in a fully unsupervised way during inference phase. Experiment results show that A2PIT is able to improve the separation performance across various numbers of speakers and effectively detect the number of speakers in a mixture. A2PIT not only allows the model to perform valid output detection in a self-supervised way without additional modules, but also achieves “fault tolerance” by the “do nothing is better than do wrong things” principle. Since the mixture itself can be treated as the output of a null separation model (i.e., perform no separation at all), the auxiliary targets force the model to generate outputs not worse than doing nothing. Moreover, the detection of invalid outputs in A2PIT can be done at frame-level based on the similarity between the outputs and the mixture, which makes it possible to perform single-pass separation and valid source detection in real-time.

Permutation Invariant Training (PIT) is a speech separation technique that aims at solving the output permutation problem in supervised learning settings, where the correct label permutation of the training targets is unknown with respect to the model outputs. PIT computes the loss between the outputs and all possible permutations of the targets, and selects the one that corresponds to the minimum loss for back-propagation. Models using PIT for training often have a fixed number of outputs, which is denoted as the number N. For the problem of separating varying numbers of sources where the actual number of sources are M≤N, N−M auxiliary targets need to be properly designed. One approach is to use low-energy random Gaussian noise as targets and detect invalid outputs by using a simple energy threshold, and it has been shown that in certain datasets this energy-based method can achieve reasonable performance.

There are two main issues in the energy-based method for invalid output detection. First, it typically cannot be jointly used with energy-invariant objective functions like SI-SDR. Second, once the detection of invalid speakers fails and the noise signals are selected as the targets, the outputs can be completely uncorrelated with any of the targets, which is undesirable for applications that require high perceptual quality or low distortion (this is referred to as the problem of lacking a “fault tolerance” mechanism for unsuccessful separation). To allow the models to use any objective functions and to have such “fault tolerance” ability, a mixture signal itself is selected as the auxiliary targets instead of random noise signals. In some embodiments, and as discussed herein, the mixture signal may be fused with hint information (i.e., speaker-attended information derived based on the listener's neural signals). For mixtures with N outputs and M<N targets, N−M mixture signals are appended to the targets and PIT is applied to find the best output permutation with respect to the targets. The A2PIT loss with the best permutation then becomes:

$${L}_{obj} = {L}_{sep} + {L}_{AE}$$

where Lₛₑₚ∈□ is the loss for the valid outputs, and L_{AE}∈□ is the auxiliary autoencoding loss for the invalid outputs with the input mixture as targets. As autoencoding is in general a much simpler task than separation, proper gradient balancing method should be applied on the two loss terms for successful training.

SI-SDR is defined as:

$$SI - SDR ( x , {x}^{⌢} ) = 10 {log}_{10} \frac{{‖ α x ‖}_{2}^{2}}{{‖ {x}^{⌢} - α x ‖}_{2}^{2}}$$

where α=\hat{x}x^{T}/xx^{T} corresponds to the optimal rescaling factor towards the estimated signal. Let α□ xx^{T}, b□ \hat{x}x^{T}, and c□ \hat{x}x^{T}. The SI-SDR can thus be expressed as:

$$SI - SDR ( x , {x}^{⌢} ) = 10 {log}_{10} ( \frac{{b}^{2} / a}{c - 2 {b}^{2} / a + {b}^{2} / a} ) = 10 {log}_{10} ( \frac{1}{ac / {b}^{2} - 1} ) = 10 {log}_{10} ( \frac{{c ( x , \hat{x} )}^{2}}{1 - {c ( x , \hat{x} )}^{2}} ) .$$

where c(x, \hat{x})□ b/\sqrt{ac}=\hat{x}x^{T}/\sqrt{(xx^{T})(\hat{x}\hat{x}^{T})} is the cosine similarity between x and \hat{x}. The scale-invariance behavior of SI-SDR can be easily observed by the nature of cosine similarity, and SI−SDR(x,\hat{x})→+∞as|c(x, \hat{x})|→1. It's easy to see that the second term in |∂SI−SDR(x, \hat{x})/∂c(x, \hat{x})| approaches infinity as |c(x, \hat{x}) approaches 1. Using it for L_{AE} may let the system to easily collapse to a local minimum which have very high performance on the auxiliary autoencoding term while failing to separate the sources. Accordingly, based on this concern, an α-skewed SI-SDR is proposed, which is defined as:

$$α SI - SDR ( x , \hat{x} ) {▯10log}_{10} ( \frac{{c ( x , \hat{x} )}^{2}}{1 + α - {c ( x , \hat{x} )}^{2}} ) ,$$

where the scale of the gradient with respect to the cosine similarity term is controlled by α≥0, and α=0 corresponds to the standard SI-SDR. For multiple-speaker utterances, α is empirically set to α=0.3 for L_{AE}, and α=0 for Lₛₑₚ. For single speaker utterances, the training target for separation is equivalent (when there is no noise) or very close (when there is noise) to the input mixture. In this case, α is also set to α=0.3 for Lₛₑₚ.

During inference phase, the detection of invalid outputs can be performed by calculating the similarity, e.g., SI-SDR score, between all outputs and the input mixture, and a threshold calculated from the training set can be used for the decision. For the “fault tolerance” mechanism, the following method is applied for selecting the valid outputs:
1. If the estimated number of outputs K is smaller than the actual number M, M-K additional outputs are randomly selected from the N-K remaining outputs.

2. If the estimated number of outputs K is larger than the actual number M, M outputs are randomly selected from the K outputs.
Another benefit for A2PIT is that it also allows frame-level detection of the invalid outputs for causal applications. Frame level detection calculates accumulated similarity starting from the first frame of the outputs, and is able to dynamically change the selected valid outputs as the similarity scores become more reliable. For streaming-based applications that require a real-time playback of the separation outputs, e.g., hearable devices, the change of the output tracks can also be easily done by switching the outputs at frame-level.

A second additional example implementation of a separation approach that can be used in conjunction with, or as an alternative to, the separation systems described in relation to FIGS. 1-13 is one based on real-time binaural speech separation with preserved spatial cues. Some separation techniques focus on generating a single-channel output for each of the target speakers, thus discarding the spatial cues needed for the localization of sound sources in space. However, preserving the spatial information is important in many applications that aim to accurately render the acoustic scene such as in hearing aids and augmented reality (AR). Therefore, in some embodiments, a further speech separation approach/algorithm is proposed that preserves the interaural cues of separated sound sources and can be implemented with low latency and high fidelity, therefore enabling a real-time modification of the acoustic scene. The present proposed approach is based on a time-domain audio separation network (TasNet), which is a single-channel time-domain speech separation system that can be implemented in real-time. Further details about example implementation of a single channel TasNet frameworks are provided in U.S. Ser. No. 16/169,194, entitled “Systems and methods for speech separation and neural decoding of attentional selection in multi-speaker environments,” the content of which is hereby incorporated by reference in its entirety. The proposed approach is a multi-input-multi-output (MIMO) end-to-end extension of the single-channel TasNet approach, in which the MIMO TasNet approach takes binaural mixed audio as input and simultaneously separates target speakers in both channels. Experimental results show that the proposed end-to-end MIMO system is able to significantly improve the separation performance and keep the perceived location of the modified sources intact in various acoustic scenes.

More particularly, in real-world multi-talker acoustic environments, humans can easily separate speech and accurately perceive the location of each speaker based on the binaural acoustic features such as interaural time differences (ITDs) and interaural level differences (ILDs). Speech processing methods aimed to modify the acoustic scene are therefore required to not only separate sound sources, but do so in a way that preserves the spatial cues needed for accurate localization of sounds. However, most binaural speech separation systems are multi-input-single-output (MISO), and hence lose the interaural cues at the output level which are important for humans to perform sound lateralization and localization. To achieve binaural speech separation as well as interaural cues preservation, the multi-input-multi-output (MIMO) proposed herein setting is used.

One issue of conventional MIMO systems is that the system latency can be perceived by humans, and the delayed playback of the separated speakers might affect the localization of the signals due to the precedence effect. To decrease the system latency while maintaining the separation quality, one solution is to use time-domain separation methods with smaller windows. Recent deep learning-based time-domain separation systems have proven their effectiveness in achieving high separation quality and decreasing the system latency. However, such systems are still MISO and their ability to perform binaural speech separation and interaural cues preservation is not fully addressed.

In the proposed approach, a multi-speaker system is formulated as a MIMO system to achieve high-quality separation and to preserve interaural cues. Based on the time-domain audio separation network, a MIMO TasNet approach is proposed that takes binaural mixture signals as input and simultaneously separates speech in both channels. The separated signals can then be directly rendered to the listener without post-processing. The MIMO TasNet exploits a parallel encoder to extract cross-channel information for mask estimation, and uses mask—and sum method to perform spatial and spectral filtering for better separation performance. Experiment results show that MIMO TasNet can performs listener-independent speech separation across a wide range of speaker angles and can preserve both ITD and ILD features with significantly higher quality than the single-channel baseline. Moreover, the minimum system latency of the systems can be less than 5 ms, showing the potentials for the actual deployment of such systems into real-world hearable devices. The proposed MIMO TasNet approach may also fuse (incorporate speaker-attended information derived from measurements of the listener's neural signals.

The problem of binaural speech separation is formulated as the separation of C sources, sᵢ^{l,r}(t)∈□^{I×T}, i=1, . . . , C from the binaural mixtures x^{l}(t), x^{r}(t) ∈□^{l×T} where the superscripts l and r denote the left and right channels, respectively. For preserving the interaural cues in the outputs, consider the case where every single source signal is transformed by a set of head-related impulse response (HRIR) filters for a specific listener:

$$\{ \begin{matrix}{s}_{i}^{l} = {\hat{s}}_{i} * {h}_{i}^{l} \\ {s}_{i}^{l} = {\hat{s}}_{i} * {h}_{i}^{r}\end{matrix} , i = 1 , ... , C$$

where ŝᵢ∈□^{l×T} is the monaural signal of source i, hᵢ^{l}, hᵢ^{r}∈□^{l×(T-T′+1)} are the pair of HRIR filters corresponding to the source i, and * represents the convolution operation. Using the HRIR-transformed signals as the separation targets forces the model to preserve interaural cues introduced by the HRIR filters, and the outputs can be directly rendered to the listener.

TasNet has been shown to achieve superior separation performance in single-channel mixtures. TasNet contains three modules: a linear encoder first transforms the mixture waveform into a two-dimensional representation; a separator estimates C multiplicative functions, and a linear decoder transforms the C target source representations back to waveforms. The TasNet pipeline incorporates cross-channel features into the single-channel model, where spatial features such as interaural phase difference (IPD) is concatenated with the mixture encoder output on a selected reference microphone for mask estimation. In various scenarios, such configurations can lead to a significantly better separation performance than the signal-channel TasNet.

The proposed MIMO TasNet uses a parallel encoder for spectro-temporal and spatial features extraction and a mask-and-sum mechanism for source separation. A primary encoder is always applied to the channel to be separated, and a secondary encoder is applied to the other channel to jointly extract cross-channel features. In other words, the sequential order of the encoders determines which channel (left of right) the separated outputs belong to. The outputs of the two encoders are concatenated (or otherwise combined) and passed to the separator, and 22C multiplicative functions are estimated for the C target speakers. C multiplicative functions are applied to the primary encoder output while the other C multiplicative functions are applied to the secondary encoder output, and the two multiplied results are then summed to create representations for C separated sources. This approach is referred to as the “mask-and-sum” mechanism to distinguish it from the other methods where only C multiplicative functions were estimated from the separation module and applied to only the reference channel. A linear decoder transforms the C target source representations back to waveforms.

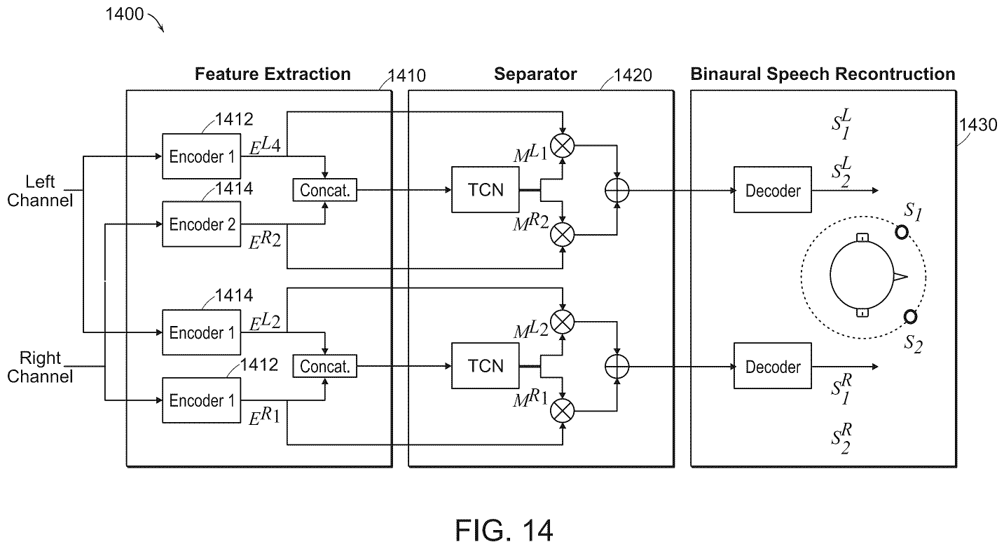

FIG. 14 is a schematic diagram of an example architecture 1400 of a multi-channel (e.g., binaural) speech separation network. The architecture 1400 includes a feature extraction section 1410 that includes multiple encoders (in the example of FIG. 14, two encoders 1412 and 1414 are depicted) which are shared by the mixture signals from both channels, and the encoder outputs for each channel are combined (e.g., concatenated, integrated, or fused in some manner), to thus preserve spatial cues, and passed to a mask estimation network. As noted, in some embodiment, hint information derived from the listener's neural signals (with such signals being indicative of the speaker(s) the listener's is attending to) may also be combined (e.g., concatenated, or integrated in some other manner) with the encoders' output and passed to a separator section 1420 (also referred to as a mask estimation network). Spectral-temporal and spatial filtering are performed by applying the masks to the corresponding encoder outputs (e.g., deriving and applying multiplicative functions derived for each group of sources from the multiple sound sources constituting the combined signal; for instance, multiplicative functions can be determined, per each of the receiving channels, for each speaker contributing to the combined signal), and the resultant outputs from the application of the multiplicative functions are summed up (e.g., on both left and right paths). Finally, the binaural separated speech is reconstructed by one or more linear decoders in a speech reconstruction section 1430. For an N-channel input, N encoders were applied to each of them, and the encoder outputs are summed to create a single representation.

When the architecture 1400 is used to perform the separation filter determination operation of, for example, the procedure 400 previously described, the combined sound signal may include in such embodiments components corresponding to multiple receiving channels (e.g., a first and second receiving channels, which may correspond to a left and a right binaural channels), and determining the separation filter may include applying multiple encoders (e.g., temporal-domain encoders) to the sound components corresponding to the multiple receiving channels, with each of the encoders applied to each of the sound components, and, for each of the multiple receiving channels, combining output components of the multiple encoders associated with respective ones of the multiple receiving channels. In such embodiments, the procedure 400 may also include deriving estimated separation functions based on the combined output components for each of the multiple receiving channels, with each of the derived estimated separation functions configured to separate the combined output components for each of the multiple receiving channels into separated sound components associated with groups (e.g., each group comprising one or more speakers) of the multiple sound sources.

Scale-invariant signal-to-distortion ratio (SI-SDR) may be used as both the evaluation metric and training objective for the present approaches. As noted, SI-SDR between a signal x∈^{lλT} and its estimate \hat{x}∈^{l×T} is defined as:

$$SI - SDR ( x , {x}^{⌢} ) = 10 {log}_{10} ( \frac{{‖ α x ‖}_{2}^{2}}{{‖ {x}^{⌢} - α x ‖}_{2}^{2}} )$$

where α=\hat{x}x_{T}/xx^{T} corresponds to the rescaling factor. Although SI-SDR is able to implicitly incorporate the ITD information, the scale-invariance property of SI-SDR makes it insensitive to power rescaling of the estimated signal, which may fail in preserving the ILD between the outputs. Thus, instead of using SI-SDR as the training objective, the plain signal-to-noise ratio (SNR) may be used instead. The SNR is defined as:

$$SNR ( x , {x}^{⌢} ) = 10 {log}_{10} ( \frac{{‖ x ‖}_{2}^{2}}{{‖ {x}^{⌢} - x ‖}_{2}^{2}} )$$

Accordingly, as discussed above, the MIMO TasNet framework, which seeks to implement real-time binaural speech separation with interaural cues preservation, uses a parallel encoder and mask-and-sum mechanism to improve performance. Experimental results show that the MIMO TasNet is able to achieve very good separation performance and has the ability to preserve interaural time difference (ITD) and interaural level difference (ILD) features.

Additional improvements may also take into account environmental noise and room reverberation, and incorporate extra microphones for obtaining more cross-channel information.

A third additional example implementation of a separation approach that can be used in conjunction with, or as an alternative to, the separation systems described in relation to FIGS. 1-14 is one based on binaural speech separation of moving speakers with preserved spatial cues. Binaural speech separation algorithms designed for augmented hearing technologies need to both improve the signal-to-noise ratio of individual speakers and preserve their perceived locations in space. The majority of binaural speech separation methods assume nonmoving speakers. As a result, their application to real-world scenarios with freely moving speakers requires block-wise adaptation which relies on short-term contextual information and limits their performance. Accordingly, a further separation approach (which like the approaches described herein may incorporate brain-informed data) for utterance-level source separation with moving speakers and in reverberant conditions is proposed. The proposed model makes use of spectral and spatial features of speakers in a larger context compared to the block-wise adaption methods. The model can implicitly track speakers within the utterance without the need for explicit tracking modules. Experimental results on simulated moving multi-talker speech show that this proposed approach can significantly outperform block-wise adaptation methods in both separation performance and preserving the interaural cues across multiple conditions, which makes it suitable for real-world augmented hearing applications. The proposed approach does not require localization and tracking modules and is thus able to preserve the spatial cues in the outputs which enables the correct localization of the separated moving source. The framework uses a binaural separation module and a binaural post enhancement module. The binaural speech separation module takes binaural mixed signals as input and simultaneously separates speech in both channels; then the left and right channel speech of each speaker are concatenated (or otherwise combined) and further enhanced by the binaural post enhancement module; the output of the binaural post enhancement module is the separated stereo sound rendered to the listener. The modules employ the TasNet framework (referred to above) that can achieve latency as low as 2 ms, and which is important for deployment in hearing devices. Experimental results show that utterance-level separation significantly outperforms the block-wise adaptation methods both in terms of signal quality and spatial cue preservation.

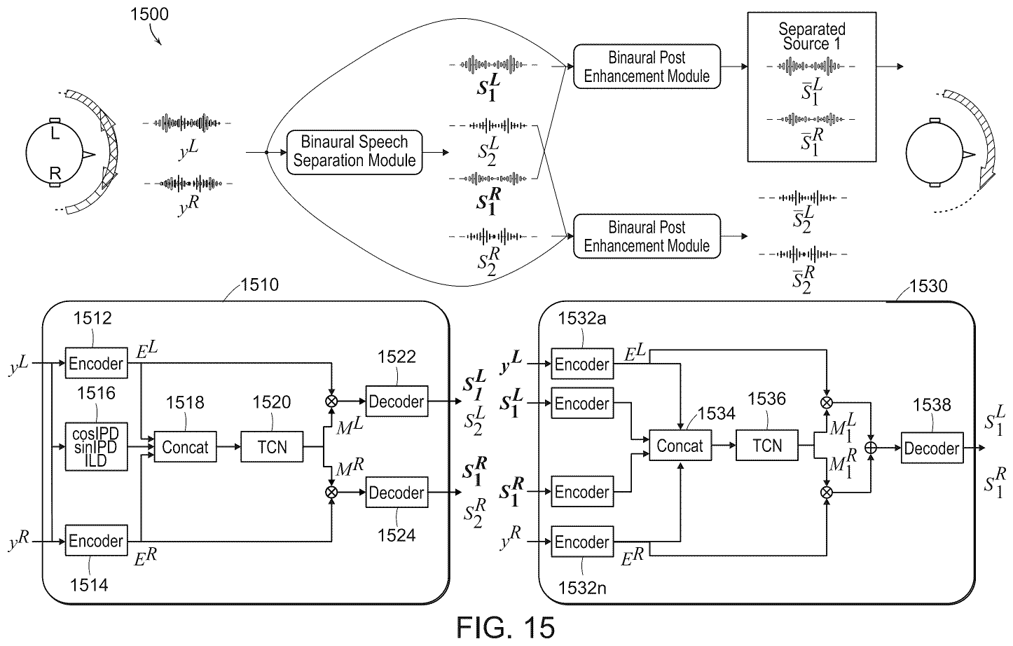

With reference to FIG. 15, a schematic diagram of an example architecture 1500 for a binaural speech separation system for moving speakers is shown. Operation of the example architecture 1500 is illustrated for two speakers (s₁ and s₂), but any number of speakers may be used in conjunction with the architecture 1500. The architecture 1500 includes a binaural speech separation section (module) 1510 and a binaural post enhancement section (or module) 1530. The binaural speech separation section 1510 simultaneously separates the speakers in each channel of the mixed input, while the section 1530 enhances each speaker individually. TasNet approaches have shown superior separation performance on various conditions, and TasNet can be implemented with causal configuration with low latency which is needed for real-time applications. In the proposed architecture 1500, a MIMO configuration is again used. As noted above in relation to the architecture 1400 of FIG. 14, the MIMO TasNet contains three steps: (1) spectral and spatial feature extraction, (2) estimation of multiplicative functions, which is similar to 2-D time-frequency masks, and (3) speech reconstruction. In the present example architecture 1500, two linear encoders transform the left- and right-channel of the mixed signals γ^{L}, γ^{R} ∈□^{T} into 2-D representations E^{L}, E^{R} ∈□^{N×H}, respectively, where N is the number of encoder basis and H is the number of time frames. To enhance the extraction of the spatial features, the interaural phase difference (IPD) information and interaural level difference (ILD) information are explicitly added as additional information/features to the outputs of the encoders 1512 and 1514. Specifically, in some embodiments, the following features are computed:

$$cos IPD = cos ( ∠ {Y}^{L} - ∠ {Y}^{R} )$$

$$sin IPD = sin ( ∠ {Y}^{L} - ∠ {Y}^{R} )$$

$$ILD = 10 {log}_{10} ( \vert {Y}^{L} \vert ∅ \vert {Y}^{R} \vert )$$

where Y^{L}, Y^{R}∈□^{N×H} are the spectrograms of Y^{L}, Y^{R}, respectively, F is the number of frequency bins, and Ø is element-wise division operation. The hop size for calculating Y^{L}, Y^{R} is the same as that for E^{L}, E^{R} to ensure they have the same number of time frames H, although the window length in the encoder is typically much shorter than that in the STFT. Finally, these cross-domain features are concatenated (or otherwise combined or integrated) by the unit 1518 (identified as “concat,” although the unit 1518 can be configured to combine the signals in other manners) into E^{M}=[E^{L}, E^{R}, cos IPD, sin IPD, ILD]∈□^{(2N+3F)×H} as the spectro-temporal and spatial-temporal features. Although not specifically shown in FIG. 15, the unit 1518 can also be configured to combine the brain-informed signal derived, for example, by the brain decoder 130, to yield E^{M}=[E^{L}, E^{R}, BIS, cos IPD, sin IPD, ILD]∈□^{(2N+3F)×H}, where BIS is the brain-informed signal generated by the decoder 130. The BIS signal may, in other embodiments, be combined with the speaker-related features/signals in other ways, and/or by other modules/units of the system 100 or 1500.

Subsequently, E^{M} is fed into a series of temporal convolutional network (TCN) blocks 1520 to estimate multiplicative function M^{L}, M^{R} ∈□^{C×N×H}, where C is the number of speakers. M^{L} and M^{R} are applied to E^{L} and E^{R}, respectively, and use one or more linear decoders 1522 and 1524 to transform the multiplied representations back to the waveforms {sᵢ^{L}}ᵢ₌ₗ^{C} and {sᵢ^{R}}ᵢ₌ₗ^{C}. Due to the permutation problem, the order of the estimated speakers in each channel cannot be pre-determined. However, a constraint that the speaker order in two channels be the same can be imposed, which is important so as to pair the left- and right-channel signals of the individual speaker in a real-time system.

The post enhancement processing section (stage) 1530 is configured to further improve the signal quality. Each stereo sound, sᵢ^{L} and sᵢ^{R} from the separation module 1510, combined with the mixed signals (y^{L}, y^{R}), is sent to a multi-input-single-output (MISO) network for post enhancement. Similar to the speech separation module, the encoder outputs (From encoders 1532a-n) are concatenated (or otherwise combined) by the unit 1534 provided to TCN blocks 1536 for estimating multiplicative functions Mᵢ^{L}, Mᵢ^{R}∈□^{2×N×H}:

$${s}_{i}^{L} = decoder ( {E}^{L} \cdot {M}_{i}^{L} [ 0 , : , : ] + {E}^{R} ▯ {M}_{i}^{L} [ 0 , : , : ] )$$

$${s}_{i}^{R} = decoder ( {E}^{L} \cdot {M}_{i}^{L} [ 1 , : , : ] + {E}^{R} ▯ {M}_{i}^{L} [ 1 , : , : ] )$$

where □ denotes element-wise multiplication. Unlike the speech separation module 1510 that only applies multiplicative functions (which is equivalent to spectral filtering), the speech enhancement module performs multiplication and sum, which is equivalent to both spectral and spatial filtering (this is similar to multichannel wiener filtering). This is therefore referred to as the mask-and-sum mechanism.

Since the input stereo sound, sᵢ^{L} and sᵢ^{R}, contains both spectral and spatial information of the speaker i, the enhancement module essentially performs informed speaker extraction without the need for permutation invariant training.

A speaker localizer (not specifically shown in FIG. 15) adopts a similar architecture as that of the speech enhancement module, but performs classification of the direction of arrival (DOA). The DOA angles are discretized into K classes. The speaker localizer takes only stereo sound, sᵢ^{L} and sᵢ^{R}, as input, concatenates (or otherwise combines) two encoders' outputs, and passes them to the TCN blocks to estimate a single-class classification matrix Vᵢ∈(0,1)^{K×H}, where “single-class” means that in each time frame, there is exactly one class labeled with 1 and all the other classes are labeled with 0. Vᵢ is split into B small chunks {Vᵢ^{b}}_{b=1}^{B}∈□^{K×Q}, where Q is the number of time frames in each chunk and B=H/Q. In each chunk the frequency of each class labeled with ‘1’ is counted, and the most frequent class is deemed as the estimated DOA for that chunk.

The signal-to-noise ratio (SNR) is used as the training objective for the speech separation and enhancement sections. SNR is sensitive to both time shift and power scale of the estimated waveform, so it's able to force the ITD and IPD to be preserved in the estimated waveform. SNR is defined as:

$$SNR ( x , {x}^{⌢} ) = 10 {log}_{10} ( \frac{{‖ x ‖}_{2}^{2}}{{‖ {x}^{⌢} - x ‖}_{2}^{2}} )$$

where \hat{x} and x are the estimated and reference signal, respectively. In the speech separation module, utterance-level permutation invariant training may be used. Thus,

$$L = {min}_{n ∈ P} {\sum }_{c = 1}^{C} SNR ( {{x}^{⌢}}_{c}^{L} - {x}_{π ( c )}^{L} ) + SNR ( {{x}^{⌢}}_{c}^{R} - {x}_{π ( c )}^{R} )$$

where P is the set of all C! permutations. The same permutation π for left- and right-channel signals assures the speaker is consistent in both channels.

When the architecture 1500 is used to perform the separation filter determination operation of, for example, the procedure 400 previously described, the combined sound signal may include, in such embodiments, representations of sound components corresponding to multiple receiving channels (e.g., a first and second receiving channels, which may correspond to a left and a right binaural channels). Determining the separation filter may include applying multiple encoders (e.g., the encoders 1512 and 1514) to the representations of sound components corresponding to the multiple receiving channels, with each of the encoders applied to each of the sound components. The determination of the separation filter also includes determining spatial features on the sounds components corresponding to the multiple receiving channels, combining (e.g., by the unit 1518 of FIG. 15) the determined spatial features with output components of the multiple encoders associated with respective ones of the multiple receiving channels, to produce a combined encoded output, deriving (e.g., by the TCN blocks 1520), based on the combined encoded output, estimated separation functions, and separating, using the estimated separation functions, the combined encoded output into separated sound components associated with groups of the multiple sound sources. In some embodiments, determining the spatial features may include determining one or more of, for example, interaural level difference (ILD) information, and/or interaural time difference (ITD) information.

In some examples, the operations performed by the architecture 1500 may further include combining the separated sound components with the representations of the sound components to produce a combined enhanced signal representation, and deriving estimated separation functions based on the combined enhanced signal representation to separate the combined enhanced signal representation into separated enhanced sound components associated with the groups of the multiple sound sources. In some additional examples, the operations performed by the architecture 1500 may further include determining, based on the separated sound components, direction of arrival of the separated sound components.

Performing the various techniques and operations described herein may be facilitated by a controller device (e.g., a processor-based computing device) that may be realized as part of a hearing aid device (that may also include a microphone and neural sensors coupled to the controller). Such a controller device may include a processor-based device such as a computing device, and so forth, that typically includes a central processor unit or a processing core. The device may also include one or more dedicated learning machines (e.g., neural networks) that may be part of the CPU or processing core. In addition to the CPU, the system includes main memory, cache memory and bus interface circuits. The controller device may include a mass storage element, such as a hard drive (solid state hard drive, or other types of hard drive), or flash drive associated with the computer system. The controller device may further include a keyboard, or keypad, or some other user input interface, and a monitor, e.g., an LCD (liquid crystal display) monitor, that may be placed where a user can access them.

The controller device is configured to facilitate, for example, the implementation of brain-informed speech separation. The storage device may thus include a computer program product that when executed on the controller device (which, as noted, may be a processor-based device) causes the processor-based device to perform operations to facilitate the implementation of procedures and operations described herein. The controller device may further include peripheral devices to enable input/output functionality. Such peripheral devices may include, for example, flash drive (e.g., a removable flash drive), or a network connection (e.g., implemented using a USB port and/or a wireless transceiver), for downloading related content to the connected system. Such peripheral devices may also be used for downloading software containing computer instructions to enable general operation of the respective system/device. Alternatively and/or additionally, in some embodiments, special purpose logic circuitry, e.g., an FPGA (field programmable gate array), an ASIC (application-specific integrated circuit), a DSP processor, a graphics processing unit (GPU), application processing unit (APU), etc., may be used in the implementations of the controller device. As noted, similar special purpose logic circuitry may also be used in the implementations of artificial learning networks. Other modules that may be included with the controller device may include a user interface to provide or receive input and output data. Additionally, in some embodiments, sensor devices such as a light-capture device (e.g., a CMOS-based or CCD-based camera device), other types of optical or electromagnetic sensors, sensors for measuring environmental conditions, etc., may be coupled to the controller device, and may be configured to observe or measure the processes and actions being monitored. The controller device may include an operating system.

Computer programs (also known as programs, software, software applications or code) include machine instructions for a programmable processor, and may be implemented in a high-level procedural and/or object-oriented programming language, and/or in assembly/machine language. As used herein, the term “machine-readable medium” refers to any non-transitory computer program product, apparatus and/or device (e.g., magnetic discs, optical disks, memory, Programmable Logic Devices (PLDs)) used to provide machine instructions and/or data to a programmable processor, including a non-transitory machine-readable medium that receives machine instructions as a machine-readable signal.

In some embodiments, any suitable computer readable media can be used for storing instructions for performing the processes/operations/procedures described herein. For example, in some embodiments computer readable media can be transitory or non-transitory. For example, non-transitory computer readable media can include media such as magnetic media (such as hard disks, floppy disks, etc.), optical media (such as compact discs, digital video discs, Blu-ray discs, etc.), semiconductor media (such as flash memory, electrically programmable read only memory (EPROM), electrically erasable programmable read only Memory (EEPROM), etc.), any suitable media that is not fleeting or not devoid of any semblance of permanence during transmission, and/or any suitable tangible media. As another example, transitory computer readable media can include signals on networks, in wires, conductors, optical fibers, circuits, any suitable media that is fleeting and devoid of any semblance of permanence during transmission, and/or any suitable intangible media.

Unless defined otherwise, all technical and scientific terms used herein have the same meaning as commonly or conventionally understood. As used herein, the articles “a” and “an” refer to one or to more than one (i.e., to at least one) of the grammatical object of the article. By way of example, “an element” means one element or more than one element. “About” and/or “approximately” as used herein when referring to a measurable value such as an amount, a temporal duration, and the like, encompasses variations of ±20% or ±10%, ±5%, or ±0.1% from the specified value, as such variations are appropriate in the context of the systems, devices, circuits, methods, and other implementations described herein. “Substantially” as used herein when referring to a measurable value such as an amount, a temporal duration, a physical attribute (such as frequency), and the like, also encompasses variations of ±20% or ±10%, ±5%, or ±0.1% from the specified value, as such variations are appropriate in the context of the systems, devices, circuits, methods, and other implementations described herein.

As used herein, including in the claims, “or” as used in a list of items prefaced by “at least one of” or “one or more of” indicates a disjunctive list such that, for example, a list of “at least one of A, B, or C” means A or B or C or AB or AC or BC or ABC (i.e., A and B and C), or combinations with more than one feature (e.g., AA, AAB, ABBC, etc.). Also, as used herein, unless otherwise stated, a statement that a function or operation is “based on” an item or condition means that the function or operation is based on the stated item or condition and may be based on one or more items and/or conditions in addition to the stated item or condition.

Although particular embodiments have been disclosed herein in detail, this has been done by way of example for purposes of illustration only, and is not intended to be limiting with respect to the scope of the appended claims, which follow. Features of the disclosed embodiments can be combined, rearranged, etc., within the scope of the invention to produce more embodiments. Some other aspects, advantages, and modifications, are considered to be within the scope of the claims provided below. The claims presented are representative of at least some of the embodiments and features disclosed herein. Other unclaimed embodiments and features are also contemplated.

What is claimed is:

1. A method for speech separation comprising:
obtaining, by a device, a combined sound signal for signals combined from multiple sound sources in an area in which a person is located;
obtaining, by the device, neural signals for the person, the neural signals being indicative of one or more target sound sources, from the multiple sound sources, the person is attentive to;
determining a separation filter based, at least in part, on the neural signals obtained for the person; and
applying, by the device, the separation filter to a representation of the combined sound signal to derive a resultant separated signal representation associated with sound from the one or more target sound sources the person is attentive to;
wherein the combined sound signal comprises sound components corresponding to multiple receiving channels, and wherein determining the separation filter comprises:
applying multiple encoders to the sound components corresponding to the multiple receiving channels, with each of the encoders applied to each of the sound components;
for each of the multiple receiving channels, combining output components of the multiple encoders associated with respective ones of the multiple receiving channels; and
deriving estimated separation functions based on the combined output components for each of the multiple receiving channels, each of the derived estimated separation functions configured to separate the combined output components for each of the multiple receiving channels into separated sound components associated with groups of the multiple sound sources.

2. The method of claim 1, wherein the multiple receiving channels comprise a first and second binaural receiving channels.

3. The method of claim 1, wherein determining the separation filter comprises:
determining based on the neural signals an estimate of an attended sound signal corresponding to the one or more target sound sources the person is attentive to; and
generating the separation filter based, at least in part, on the determined estimate of the attended sound signal.

4. The method of claim 3, wherein determining the estimate of the attended sound signal comprises:
determining, using a learning process, an estimated target envelope for the one or more target sound sources the person is attentive to, the estimated target envelope being combined with the output components of the multiple encoders.

5. The method of claim 1, wherein obtaining the neural signals for the person comprises measuring the neural signals according to one or more of: invasive intracranial electroencephalography (iEEG) recordings, non-invasive electroencephalography (EEG) recordings, functional near-infrared spectroscopy (fNIRS) recordings, or recordings captured with subdural or brain-implanted electrodes.

6. The method of claim 1, wherein deriving the estimated separation functions comprises:
processing the combined output components for each of the multiple receiving channels with respective one or more temporal convolutional network (TCN) blocks to estimate multiplicative functions that are applied to the output components of the multiple encoders associated with respective ones of the multiple receiving channels.

7. The method of claim 1, further comprising:
reconstructing the separated sound components, using linear decoders, into binaural signals associated with selected one or more of the groups of the multiple sound sources.

8. A system comprising:
at least one microphone to obtain a combined sound signal for signals combined from multiple sound sources in an area in which a person is located;
one or more neural sensors to obtain neural signals for the person, the neural signals being indicative of one or more target sound sources, from the multiple sound sources, the person is attentive to; and
a controller in communication with the at least one microphone and the one or more neural sensors, the controller configured to:
determine a separation filter based, at least in part, on the neural signals obtained for the person; and
apply the separation filter to a representation of the combined sound signal to derive a resultant separated signal representation associated with sound from the one or more target sound sources the person is attentive to;
wherein the combined sound signal comprises sound components corresponding to multiple receiving channels, and wherein the controller configured to determine the separation filter is configured to:
apply multiple encoders to the sound components corresponding to the multiple receiving channels, with each of the encoders applied to each of the sound components;
combine, for each of the multiple receiving channels, output components of the multiple encoders associated with respective ones of the multiple receiving channels; and
derive estimated separation functions based on the combined output components for each of the multiple receiving channels, each of the derived estimated separation functions configured to separate the combined output components for each of the multiple receiving channels into separated sound components associated with groups of the multiple sound sources.

9. The system of claim 8, wherein the multiple receiving channels comprise a first and second binaural receiving channels.

10. The system of claim 8, wherein the controller configured to determine the separation filter is configured to:
determine based on the neural signals an estimate of an attended sound signal corresponding to the one or more target sound sources the person is attentive to; and
generate the separation filter based, at least in part, on the determined estimate of the attended sound signal.

11. The system of claim 10, wherein the controller configured to determine the estimate of the attended sound signal is configured to:
determine, using a learning process, an estimated target envelope for the one or more target sound sources the person is attentive to, the estimated target envelope being combined with the output components of the multiple encoders.

12. The system of claim 8, wherein the one or more neural sensors to obtain neural signals for the person comprise at least one sensor to measure the neural signals according to one or more of: invasive intracranial electroencephalography (iEEG) recordings, non-invasive electroencephalography (EEG) recordings, functional near-infrared spectroscopy (fNIRS) recordings, or recordings captured with subdural or brain-implanted electrodes.

13. The system of claim 8, wherein the controller configured to derive the estimated separation functions is configured to:
process the combined output components for each of the multiple receiving channels with respective one or more temporal convolutional network (TCN) blocks to estimate multiplicative functions that are applied to the output components of the multiple encoders associated with respective ones of the multiple receiving channels.

14. The system of claim 8, wherein the controller is further configured to:
reconstruct the separated sound components, using linear decoders, into binaural signals associated with selected one or more of the groups of the multiple sound sources.

15. Non-transitory computer readable media comprising computer instructions executable on a processor-based device to:
obtain a combined sound signal for signals combined from multiple sound sources in an area in which a person is located;
obtain neural signals for the person, the neural signals being indicative of one or more target sound sources, from the multiple sound sources, the person is attentive to;
determine a separation filter based, at least in part, on the neural signals obtained for the person; and
apply the separation filter to a representation of the combined sound signal to derive a resultant separated signal representation associated with sound from the one or more target sound sources the person is attentive to;
wherein the combined sound signal comprises sound components corresponding to multiple receiving channels, and wherein the computer instructions to determine the separation filter comprise one or more computer instructions to:
apply multiple encoders to the sound components corresponding to the multiple receiving channels, with each of the encoders applied to each of the sound components;
for each of the multiple receiving channels, combine output components of the multiple encoders associated with respective ones of the multiple receiving channels; and
derive estimated separation functions based on the combined output components for each of the multiple receiving channels, each of the derived estimated separation functions configured to separate the combined output components for each of the multiple receiving channels into separated sound components associated with groups of the multiple sound sources.

16. The computer readable media of claim 15, wherein the multiple receiving channels comprise a first and second binaural receiving channels.

17. The computer readable media of claim 15, wherein the computer instructions to determine the separation filter include one or more instructions to:
determine based on the neural signals an estimate of an attended sound signal corresponding to the one or more target sound sources the person is attentive to; and
generate the separation filter based, at least in part, on the determined estimate of the attended sound signal.

18. The computer readable media of claim 17, wherein the one or more instructions to determine the estimate of the attended sound signal include additional one or more instructions to:
determine, using a learning process, an estimated target envelope for the one or more target sound sources the person is attentive to, the estimated target envelope being combined with the output components of the multiple encoders.

19. The computer readable media of claim 15, wherein the computer instructions to obtain the neural signals for the person include one or more instructions to measure the neural signals according to one or more of: invasive intracranial electroencephalography (iEEG) recordings, non-invasive electroencephalography (EEG) recordings, functional near-infrared spectroscopy (fNIRS) recordings, or recordings captured with subdural or brain-implanted electrodes.

20. The computer readable media of claim 15, wherein the computer instructions comprise additional instructions to:
reconstruct the separated sound components, using linear decoders, into binaural signals associated with selected one or more of the groups of the multiple sound sources.
